(19) United States
(12) Patent Application Publication
(10) Pub. No.: US 2023/0170071 A1
(43) Pub. Date: Jun. 1, 2023

(54) SYSTEMS AND METHODS FOR PROVIDING PERSONALIZED NUTRITIONAL INFORMATION AND RECOMMENDATIONS

(71) Applicant: Heali AI Corp., Santa Monica, CA (US)

(72) Inventors: Kyle Dardashti, Santa Monica, CA (US); William Brendel, Reno, NV (US); Renee Fitton, West Hollywood, CA (US); Matthew Helms, Los Angeles, CA (US); Parker Saussy, Los Angeles, CA (US); Jennifer Kuca, Los Angeles, CA (US)

(21) Appl. No.: 18/070,724

(22) Filed: Nov. 29, 2022

Related U.S. Application Data
(60) Provisional application No. 63/284,255, filed on Nov. 30, 2021.

(51) Int. Cl. G16H 20/60; G16H 50/20

(52) U.S. Cl. CPC G16H 20/60; G16H 50/20

(57) ABSTRACT

The invention is directed to systems and methods that utilize artificial intelligence (AI) and big data analytics to autonomously provide personalized and precise nutritional information and recommendations to a user.

### CROSS-REFERENCE TO RELATED APPLICATIONS

This application claims priority to, and the benefit of, U.S. Provisional Application No. 63/284,255, filed Nov. 30, 2021, the content of which is incorporated by reference herein in its entirety.

### TECHNICAL FIELD

The invention generally relates to nutrition, and, more particularly, to systems and methods for providing personalized nutritional information and recommendations to a user based, at least in part, on artificial intelligence (AI) and big data analytics.

### BACKGROUND

Individuals seeking to pursue a healthier lifestyle have been inundated with dietary choices and options. Furthermore, deciding what foods to purchase and how to prepare such foods proves to be a challenge.

Historically, nutrition tracking required users to manually record the food they consumed. Users generally did this by writing consumed items in a notepad and referring to general food information to determine the corresponding nutrient content. This process required time and continued effort from the user, which could lead to omissions or abandoning the practice. To save time, users sometimes limit themselves to tracking only select nutrients such as fat or calories and therefore would not have a full understanding of their food's comprehensive nutrient content. Additionally, manual tracking required users to do their own calculations of total nutrient intake, which raised the risk of errors. Users also had to independently research and seek foods that optimized their nutrient intake, which could yield suboptimal results due a user's limited exposure to and knowledge of all possible foods and their nutrient contents.

As such, dietary tracking and related software applications are becoming increasingly popular as a means to monitor the nutritional value of the food products being ingested by individuals and to assist those individuals in making healthier food choices.

For example, in certain software applications, an individual directly inputs the food products that were consumed for each meal into the software application, which, in turn, pulls certain nutritional information about the consumed food products, including the number and types of calories associated with the consumed food products, from a database. In this way, the individual can then keep track of the number of calories being consumed, as well as the amount of protein, fat, and carbohydrates in those food products, to thereby make healthier choices and maintain healthy eating habits. In other exemplary software applications, an individual may use their smartphone to scan a packaged food product's bar code or to search for a food product. The software application will then, based on data stored in an associated database, display a letter grade for that packaged food product to allow the individual to see if they are making a healthy food choice.

While conventional nutrition-based services, such as the software applications described above, provide a user with some information regarding their dietary choices, such services remain limited as a result of inadequate and poor data. In particular, such services lack an adequate understanding of overall nutritional characteristics of ingredients, prepared foods, food products, and recipes as a result of missing and/or hidden data that is required for determining and fully appreciating the nutritional characteristics of ingredients, prepared foods, food products, and recipes.

### SUMMARY

The present invention recognizes the drawbacks of current health related services, particularly dietary tracking software applications, and provides a nutritional guidance system to address such drawbacks.

In particular, aspects of the invention may be accomplished by using a nutrition and dietary management platform providing a portal or user interface (UI) with which a user may interact via an associated computing device, such as a smartphone or tablet or PC. For example, in one embodiment, the platform is provided via a mobile application (i.e., an “app”) that a user can access and interact with via their smartphone. The platform provides a suite of features that allows for a user to obtain personalized nutritional information and recommendations based on artificial intelligence (AI) and big data analytics.

In particular, a user may initially create profile in which they provide basic information (i.e., name, age, gender, contact information, etc.). The platform further allows for a user to provide other attributes, including, but not limited to, biometric data (i.e., DNA and/or user microbiome data), demographic data, user food and/or symptom logs, user medical conditions, user lab results, etc.). Additionally, the platform allows for a user to set certain preferences, such as a preferred dietary lifestyle (i.e., vegan, vegetarian, paleo, ketogenic, gluten free, dairy free, Mediterranean, etc.), as well as preferred nutrient intake/avoidance, such as less of a specific nutrient or vitamin (e.g., less calories, less carbohydrates, less fat, less sugar, less sodium, less cholesterol, etc.) or more of a specific nutrient or vitamin (e.g., more protein, more fiber, more iron, more calcium, etc.).

Upon a user establishing their attributes and preferences, the nutritional guidance system of the present invention is able to determine optimal eating plan for the user. More specifically, the nutrition and dietary management platform is configured to provide personalized nutritional information and recommendations to a user based, at least in part, on artificial intelligence (AI) and big data analytics. For example, such personalized nutritional information and recommendations are based on the processing sets of data, including a food graph and a user graph, which are generated using a data hierarchy structure leveraging food attributes (i.e., nutrition facts, food tags, etc.) and user attributes (i.e., the user attributes previously described herein), respectively. The food graph may generally include a set of data associated with various food items, such as ingredients, products, recipes, and the like, which may be obtained from a database that has been populated with millions of food items and associated information. Such food items and associated information is generally acquired from various data sources (including publicly available data sources for any given food item). The user graph may generally include a set of data associated with the user attributes. Upon generating food and user graphs, the system is able to programmatically build mappings between the food graph and the user graph using AI-based techniques, such as machine learning, natural language processing, reinforcement learning, and data mining techniques. Upon establishing mappings between the food graph and user graph, the system is configured to autonomously generate one or more dietary predictions, dietary recommendations, and/or dietary modifications personalized to a given user based on their attributes and preferences.

As described herein, the nutrition and dietary management platform is configured to provide a user with personalized nutritional information and recommendations based on the AI and big data analytics described above. For example, in some instances, the platform is configured to automatically provide a user with recommended food items, recipes, meal plans, and the like, based on a user set preference (i.e., the user may set a preference for the platform to automatically generate a daily, weekly, and/or monthly suggested meal plan). In some embodiments, a user may directly interact with the platform to find a given food item, recipe, or meal plan. For example, a user may use their smartphone to take a photograph of a menu item or scan a food item barcode. In turn, the system of the present invention is configured to identify the menu item (and all related food items in that menu item) or the food item and provide the user (via the platform interface) with an indication as to whether such foods adhere to the individualized nutritional preferences and, in the event that such foods do not adhere to the preferences, the system is configured to provide alternative options to choose.

Accordingly, the nutritional guidance system of the present invention, notably the nutrition and dietary management platform, better connects a user with a vast amount of nutritional data, thereby making it easier for users to make better dietary choices for every meal. The platform provides an intuitive means allowing for a user to search through a large database of ingredients to find the food items, meals, and recipes that best match a user's preferences and fit within their lifestyle. As such, the system of the present invention is able to assist a user with managing their diet and further support therapeutic nutritional plans such as keto, gluten-free, and Low-FODMAP with tools that may assist in treating or managing chronic diseases with optimal nutrition.

One aspect of the present invention is directed to a system for providing personalized nutrition services. The system includes a computing system configured to communicate with one or more user-associated computing devices over a network. The computing system comprising a hardware processor coupled to non-transitory, computer-readable memory containing instructions executable by the processor to cause the computing system to perform a number of operations. In particular, the computing system is configured to generate a food graph based on attributes associated with food items extracted from one or more data sources. The computing system is further configured to receive, from a computing device, user input based on user interaction with a graphical user interface (GUI) of the computing device and generate a user graph based, at least in part, on said user input. The user input comprises user attributes including, but not limited to, user biometric data, user demographic data, user food and/or symptom logs, user medical conditions, and user lab results.

In turn, the computing system is configured to command a trained artificial intelligence (AI) engine to build mappings between the food graph and the user graph. In turn, the computing system is configured to autonomously generate one or more dietary predictions, dietary recommendations, and/or dietary modifications based, at least in part, on the mappings established between the food and user graphs. The one or more dietary predictions, dietary recommendations, and dietary modifications include at least one of: a personalized diet for the user; a meal plan for the user; food recommendations for the user; restaurant recommendations for the user; recipe recommendations for the user; and product recommendations for the user.

In some embodiments, the computing system is further configured to autonomously tag food items with at least some of the attributes based on an initial set of attributes for the food items in the food graph.

In some embodiments, the computing system may further be configured to detect, via the trained artificial intelligence engine, missing, hidden, and/or incorrect attribute values. For example, the computing system may be configured to add or correct the missing, hidden, and/or incorrect attribute values.

In some embodiments, the computing system is further configured to generate food similarity metrics between the food items in the food graph based on the attributes.

In some embodiments, the computing system is further configured to generate user similarity metrics based on the user graph.

Another aspect of the present invention is directed to a method for providing personalized nutrition services. The method includes generating a food graph based on attributes associated with food items extracted from one or more data sources. The method further includes receiving, from a computing device, user input based on user interaction with a graphical user interface (GUI) of the computing device and generating a user graph based on said user input. The user input comprises user attributes including, but not limited to, user biometric data, user demographic data, user food and/or symptom logs, user medical conditions, and user lab results.

The method further includes commanding a trained artificial intelligence (AI) engine to build mappings between the food graph and the user graph. The method further includes autonomously generating one or more dietary predictions, dietary recommendations, and/or dietary modifications based, at least in part, on the mappings established between the food and user graphs. The one or more dietary predictions, dietary recommendations, and dietary modifications may include, but are not limited to, a personalized diet for the user, a meal plan for the user, food recommendations for the user, restaurant recommendations for the user, recipe recommendations for the user, and product recommendations for the user.

In some embodiments, the method further comprises autonomously tagging food items with at least some of the attributes based on an initial set of attributes for the food items in the food graph.

In some embodiments, the method further comprises detecting, via the trained artificial intelligence engine, missing, hidden, and/or incorrect attribute values. The method may further include adding or correcting the missing, hidden, and/or incorrect attribute values.

In some embodiments, the method further comprises generating food similarity metrics between the food items in the food graph based on the attributes.

In some embodiments, the method further comprises generating user similarity metrics based on the user graph.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a block diagram illustrating one embodiment of an exemplary system for providing a nutritional and dietary guidance services consistent with the present disclosure.

FIG. 2 is a block diagram illustrating the nutrition and dietary management platform of FIG. 1 in greater detail.

FIG. 3 is a block diagram illustrating an exemplary computing system for implementing one or more servers for running the nutrition and dietary management platform consistent with the present disclosure.

FIG. 4 is a block diagram illustrating at least one embodiment of a client device (i.e., mobile device) for communicating with the nutrition and dietary management platform and providing an interface upon which the user can interact so as to obtain personalized nutritional information and recommendations via the nutrition and dietary management platform.

FIG. 5 is a block diagram illustrating an exemplary computing environment for implementing the nutrition and dietary management platform consistent with the present disclosure.

FIG. 6 is a block diagram illustrating inputting of reference data (i.e., training data sets) into the AI engine of the nutrition and dietary management platform.

FIG. 7 shows an AI engine according to certain embodiments of the present disclosure.

FIG. 8 is a flow diagram illustrating the overall process for providing nutritional and dietary guidance services and solving precision nutrition problems via the nutrition and dietary management platform consistent with the present disclosure.

FIGS. 9A and 9B are exemplary depictions of a food graph generated via a graph generator module consistent with the present disclosure, wherein the food graph nodes represent food items (see FIG. 9A) and a relationship between food items is captured on edges of the food graph (see FIG. 9B).

FIGS. 10A and 10B are exemplary depictions of a user graph generated via a graph generator module consistent with the present disclosure, wherein the user graph nodes represent users with attributes (see FIG. 10A) and a relationship between attributes is captured on edges of the user graph (see FIG. 10B).

FIG. 11 is an exemplary depiction of mappings established between the food graph and the user graph based on AI-based techniques, including, but not limited to, machine learning, natural language processing, reinforcement learning, and data mining techniques.

FIG. 12 is a flow diagram illustrating the overall process for populating a database with food items and organizing/categorizing various aspects of a given food item via the nutrition and dietary management platform consistent with the present disclosure.

FIG. 13 is a flow diagram illustrating the general process in which a user can establish their dietary preferences via the nutrition and dietary management platform.

FIGS. 14 and 15 are screenshots of an interface on a mobile device associated with the nutritional and dietary guidance services provided by the nutrition and dietary management platform of the present disclosure, in which a user is navigating through the initial onboarding process, including selected specific dietary needs or desired dietary lifestyle.

FIG. 16 is a screenshot of an exemplary interface providing a summary of a user's various preferences with regard to their medical nutrition therapy and/or preferred dietary lifestyle, which further may include preferred nutrient intake and avoidance.

FIG. 17 is a screenshot of an exemplary interface providing the user with different food options that provide specific benefits (i.e., foods and recipes for addressing digestion-related issues, promoting muscle growth, improving bone structure and strength, improving eyesight, and the like).

FIGS. 18 and 19 are screenshots illustrating a user utilizing their mobile device to scan a food item barcode (see FIG. 18) or capture an image of a menu item (see FIG. 19), wherein the system of the present disclosure is configured to identify the scanned food item or specific meals on the menu and provide the user with nutritional information associated therewith.

FIG. 20 is a screenshot of an interface on a mobile device associated with the nutritional and dietary guidance services provided by the nutrition and dietary management platform of the present disclosure, in which a user is presented with an indication as to the degree to which a food item or recipe or meal matches a user's personalized nutritional information and recommendations.

FIG. 21 is a screenshot of the interface on a user's mobile device, in which a user is presented with a nutrition profile of a given food item of interest.

FIGS. 22A-22H are screenshots of the interface on a user's mobile device illustrating various messages associated with personalized nutritional information and recommendations via the nutrition and dietary management platform.

FIG. 22A shows a message in which a user is advised that a certain recipe or meal may contain red meat, a broad category food items, which may include certain types of red meat that the user should avoid or has excluded based on their user attributes and preferences.

FIG. 22B shows a message in which a user is advised that a specific food item in a given recipe or meal is associated with a food item that the user should avoid or has excluded from their diet based on their user attributes and preferences.

FIG. 22C shows a message in which a user is advised that a specific food item is similarly matched with a food item that the user should avoid or has excluded from their diet based on their user attributes and preferences.

FIG. 22D shows a message in which a user is advised that a specific quantity of food item is recommended for a user preferred dietary lifestyle or therapeutic plan.

FIG. 22E shows a message in which a user is advised that certain forms a of specific food item is not recommended for a user preferred dietary lifestyle or therapeutic plan.

FIG. 22F shows a message in which a user is cautioned that certain nutrients (i.e., vitamins, minerals, or the like) may have a high correlation to their food item source that the user should avoid or has excluded from their diet based on their user attributes and preferences.

FIG. 22G shows a message in which a user is advised that a specific food item or meal may contain certain ingredients/sub-products that the user should avoid or has excluded from their diet based on their user attributes and preferences.

FIG. 22H shows a message in which a user is provided with potential substitute ingredients to be used so as to avoid any potential ingredients/sub-products identified that the that the user should avoid or has excluded from their diet based on their user attributes and preferences.

FIGS. 23A and 23B show an interface in which a user can plan their meals for any given day (see FIG. 23A) and further select specific food items to be purchased and delivered via a participating third-party service (see FIG. 23B).

FIG. 24 shows an exemplary interface providing a user with the ability to save recipes.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present invention is directed to a nutritional guidance system that includes a nutrition and dietary management platform with which users can interact to obtain personalized nutritional information and recommendations based on artificial intelligence (AI) and big data analytics.

As previously described herein, there is a data problem associated with the understanding of nutritional characteristics of ingredients, prepared foods, food products, and recipes. Such a problem exists because there is missing and/or hidden data that is required for determining and fully understanding the nutritional characteristics of ingredients, prepared foods, food products, and recipes. The systems and methods of the present disclosure solve such problems and are able to further address the issues associated with precision nutrition and personalized nutrition for the masses.

Precision nutrition is a credible emerging area of research under the umbrella of precision medicine. Precision medicine seeks to improve the personalized treatment of diseases, and precision nutrition is specific to dietary intake. Both develop interventions to prevent or treat chronic diseases based on a person's unique characteristics like DNA, race, gender, health history, and lifestyle habits. Both aim to provide safer and more effective ways to prevent and treat disease by providing more accurate and targeted strategies. Precision nutrition assumes that each person may have a different response to specific foods and nutrients, so that the best diet for one individual may look very different than the best diet for another. Precision nutrition also considers the microbiome (i.e., the trillions of bacteria in the human body that play a key role in various daily internal operations). What types and how much bacteria a person has is unique to each individual. As such, an individual's diet can determine which types of bacteria live in their digestive tract. Furthermore, the types of bacteria an individual has may be determinative of how that individual breaks down certain foods and what types of foods are most beneficial for their body.

The nutrition and dietary management platform provides an interactive interface with which a user may interact, specifically via their smartphone or tablet PC, for example. The platform provides a suite of features that allows for a user to provide specific attributes, including, but not limited to, biometric data (i.e., DNA and/or user microbiome data), demographic data, user food and/or symptom logs, user medical conditions, user lab results, etc.), as well as establish certain preferences, such as a preferred dietary lifestyle and/or preferred nutrient intake/avoidance. Accordingly, the systems and methods of the present invention are able to provide nutritional and dietary information and recommendations based, at least in part, on a given user's attributes, such as a user's biometrics (i.e., DNA, microbiome, and metabolic response to specific foods or dietary patterns) to determine the most effective eating plan to prevent or treat disease.

For example, such personalized nutritional and dietary information and recommendations are based on the processing sets of data, including a food graph and a user graph, which are generated using a data hierarchy structure leveraging food attributes (i.e., nutrition facts, food tags, etc.) and user attributes (i.e., the user attributes previously described herein), respectively. The food graph may generally include a set of data associated with various food items, such as ingredients, products, recipes, and the like, which may be obtained from a database that has been populated with millions of food items and associated information. Such food items and associated information is generally acquired from various data sources (including publicly available data sources for any given food item). The user graph may generally include a set of data associated with the user attributes. Upon generating food and user graphs, the system is able to programmatically build mappings between the food graph and the user graph using AI-based techniques, such as machine learning, natural language processing, reinforcement learning, and data mining techniques. Upon establishing mappings between the food graph and user graph, the system is configured to autonomously generate one or more dietary predictions, dietary recommendations, and/or dietary modifications personalized to a given user based on their attributes and preferences.

Accordingly, the nutritional guidance system of the present invention, notably the nutrition and dietary management platform, better connects a user with a vast amount of nutritional data, thereby making it easier for users to make better dietary choices for every meal. The platform provides an intuitive means allowing for a user to search through a large database of ingredients to find the food items, meals, and recipes that best match a user's preferences and fit within their lifestyle. As such, the system of the present invention is able to assist a user with managing their diet and further support therapeutic nutritional plans with tools that may assist in treating or managing chronic diseases with optimal nutrition.

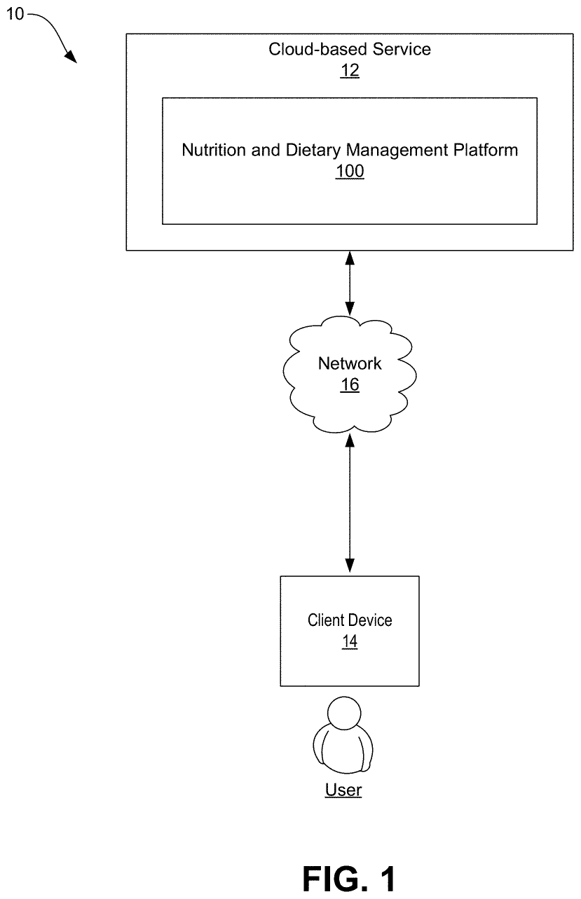

FIG. 1 is a block diagram illustrating one embodiment of an exemplary system 10 for providing a nutritional and dietary guidance services. As shown, system 10 includes a nutrition and dietary management platform 100 embodied on an internet-based computing system/service. For example, as shown, the nutrition and dietary management platform 100 may be embodied on a cloud-based service 12, for example. The nutrition and dietary management platform 100 is configured to communicate and share data, specifically nutrition and dietary-related data, with one or more users via client devices 14 over a network 16.

The network 16 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the mobile devices 14 and/or between the mobile devices 14 and the cloud-based service 12, may be, in whole or in part, a wired connection.

The network 16 may be any network that carries data. Non-limiting examples of suitable networks that may be used as network 16 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G), fifth generation (5G) cellular-based data communication technologies, Bluetooth radio, Near Field Communication (NFC), the most recently published versions of IEEE 802.11 transmission protocol standards, other networks capable of carrying data, and combinations thereof In some embodiments, network 16 is chosen from the internet, at least one wireless network, at least one cellular telephone network, and combinations thereof. As such, the network 16 may include any number of additional devices, such as additional computers, routers, and switches, to facilitate communications. In some embodiments, the network 16 may be or include a single network, and in other embodiments the network 16 may be or include a collection of networks.

The nutrition and dietary management platform 100 is configured to communicate and share data with client devices 14 associated with a consumer. Accordingly, the computing device 14 may be embodied as any type of device for communicating with the nutrition and dietary management platform 100 and cloud-based service 12, and/or other user devices over the network 16. For example, the client device may be embodied as, without limitation, a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure. In the embodiments described here, the client device 14 is generally embodied as a smartphone or tablet. However, it should be noted that one or more devices 14 may include a computer, a desktop computer, a personal computer (PC), a tablet computer, a laptop computer, a notebook computer, and the like.

It should be noted that embodiments of the system 10 of the present disclosure include computer systems, computer operated methods, computer products, systems including computer-readable memory, systems including a processor and a tangible, non-transitory memory configured to communicate with the processor, the tangible, non-transitory memory having stored instructions that, in response to execution by the processor, cause the system to perform steps in accordance with the disclosed principles, systems including non-transitory computer-readable storage medium configured to store instructions that when executed cause a processor to follow a process in accordance with the disclosed principles, etc.

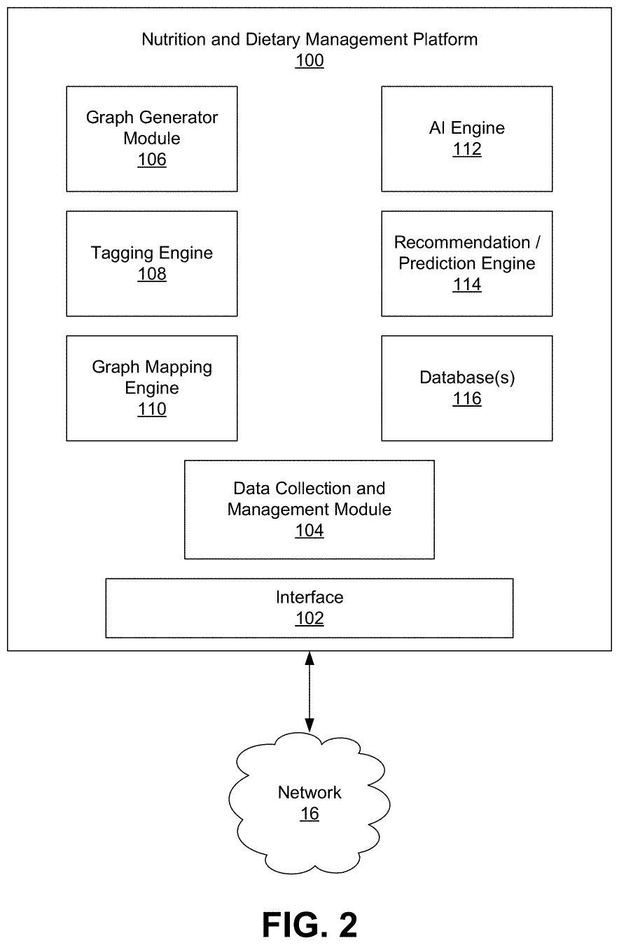

FIG. 2 is a block diagram illustrating the nutrition and dietary management platform 100 in greater detail. As shown, the nutrition and dietary management platform 100 may include an interface 102, a data collection and management module 104, a graph generator module 106, a tagging engine 108, a graph mapping engine 110, an AI engine 112, a recommendation/prediction engine 114, and various databases 116 for storage of data. For example, the databases 116 may include a user database for storing user profiles, including basic user information, user attributes, and user preferences. The databases 116 may further include a food item database for storing millions of food items and associated information. It should be noted that such food items and associated information is generally acquired from various data sources (including publicly available data sources for any given food item). The databases 116 may further include a food graph database and a user graph database (for storing generated food and user graphs), database(s) for storage of established mappings between food and user graphs, and the like. The data collection and management module 104 may be configured to communicate and exchange data with each of the databases. Accordingly, the databases 116 may include, for example, data associated ingredients, food items, recipes, prepared foods, and associated attributes and tags, user information and attributes, food graphs, user graphs, diet information and attributes, training data for training artificial intelligence algorithms and machine learning models, test/validation data for testing trained artificial intelligence and machine learning models, parameters and/or coefficients for trained machine learning models, outputs of machine learning models, and/or any other data that can be used for implementing embodiments of the platform 100.

The interface 102 generally allows a user to access data on the nutrition and dietary management platform 100, via a mobile software application, for example, provided on a mobile device or via a web-based portal. For example, upon accessing a mobile software application, the interface 102 may be presented to the user via their device 14, in which the user may navigate a dashboard or standard platform interface so as to view data (stored in one or more of the databases) and to further utilize the platform 100 for food item searching and selection and for receiving personalized nutritional information and recommendations, as described in greater detail herein. The function of each of the various modules and engines of the platform 100 will be described in greater detail herein.

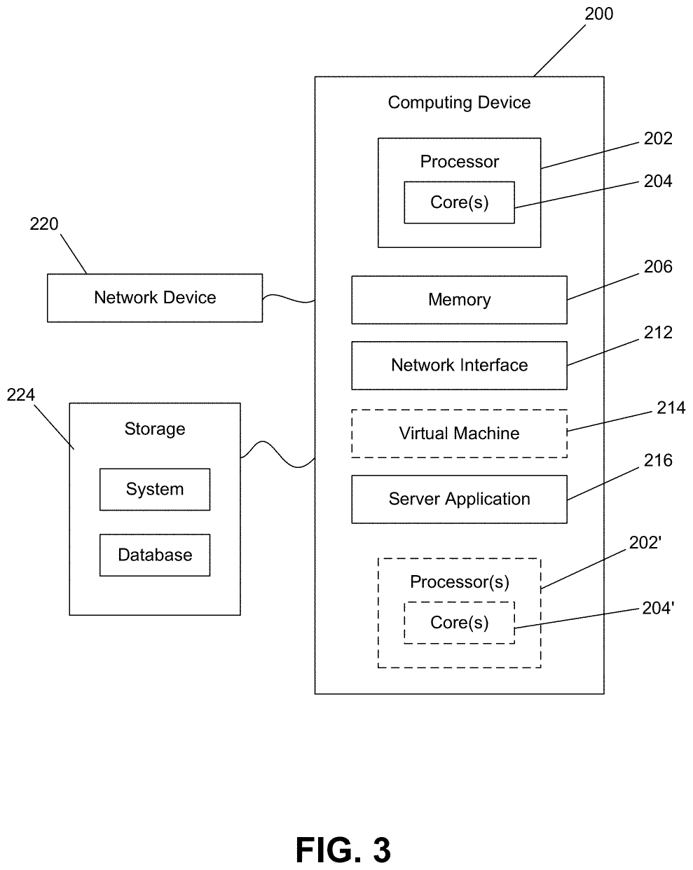

FIG. 3 is a block diagram illustrating an exemplary computing system 200 for implementing one or more servers for running the nutrition and dietary management platform 100 consistent with the present disclosure. In the present embodiment, the computing system 200 is configured as a server that is programmed and/or configured to execute one of more of the operations and/or functions for embodiments of the platform 100 described herein and to facilitate communication with the user devices via the user interface. The computing system 200 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more solid state drives), and the like. For example, memory 206 included in the computing system 200 can store computer-readable and computer-executable instructions or software for implementing exemplary embodiments of the components/modules of the platform 100 or portions thereof. The computing system 200 also includes configurable and/or programmable processor 202 and associated core 204, and optionally, one or more additional configurable and/or programmable processor(s) 202′ (e.g., central processing unit, graphical processing unit, etc.) and associated core(s) 204′ (for example, in the case of computer systems having multiple processors/cores), for executing computer-readable and computer-executable instructions or software stored in the memory 206 and other programs for controlling system hardware. Processor 202 and processor(s) 202′ may each be a single core processor or multiple core (204 and 204′) processor.

Virtualization may be employed in the computing system 200 so that infrastructure and resources in the computing device may be shared dynamically. One or more virtual machines 214 may be provided to handle a process running on multiple processors so that the process appears to be using only one computing resource rather than multiple computing resources. Multiple virtual machines may also be used with one processor.

Memory 206 may include a computer system memory or random access memory, such as DRAM, SRAM, EDO RAM, and the like. Memory 206 may include other types of memory as well, or combinations thereof.

The computing system 200 may include or be operatively coupled to one or more data storage devices 224, such as a hard-drive, CD-ROM, mass storage flash drive, or other computer readable media, for storing data and computer-readable instructions and/or software that can be executed by the processing device 202 to implement exemplary embodiments of the components/modules described herein with reference to the platform 100.

The computing system 200 can include a network interface 212 configured to interface via one or more network devices 220 with one or more networks, for example, a Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections (including via cellular base stations), controller area network (CAN), or some combination of any or all of the above. The network interface 212 may include a built-in network adapter, network interface card, PCMCIA network card, card bus network adapter, wireless network adapter, USB network adapter, modem or any other device suitable for interfacing the computing system 200 to any type of network capable of communication and performing the operations described herein. While the system device 200 depicted in block diagram is implemented as a server, exemplary embodiments of the computing system 200 can be any computer system, such as a workstation, desktop computer or other form of computing or telecommunications device that is capable of communication with other devices either by wireless communication or wired communication and that has sufficient processor power and memory capacity to perform the operations described herein.

The computing system 200 may run any server operating system or application 216, such as any of the versions of server applications including any Unix-based server applications, Linux-based server application, any proprietary server applications, or any other server applications capable of running on the computing system 200 and performing the operations described herein. An example of a server application that can run on the computing device includes the Apache server application.

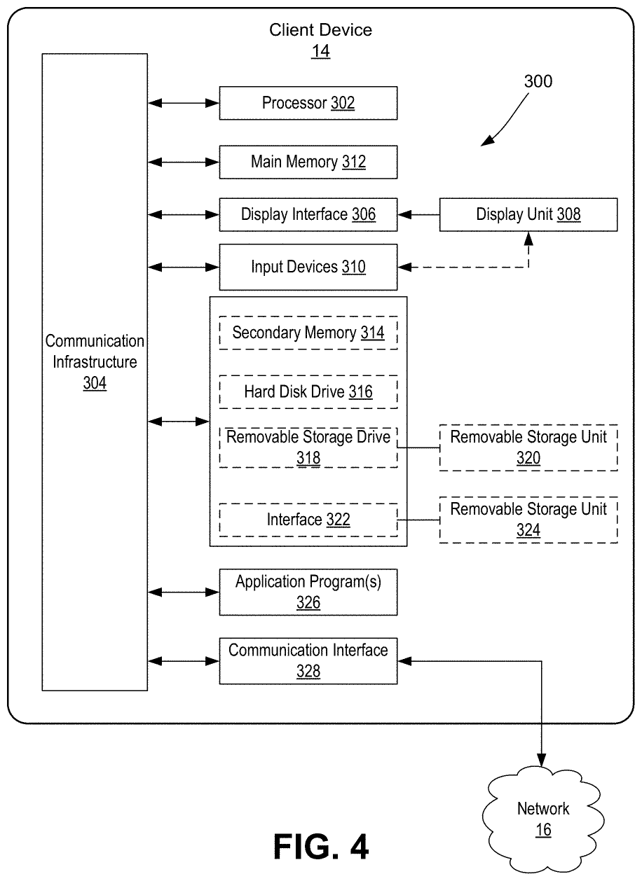

FIG. 4 is a block diagram illustrating at least one embodiment of a client device (i.e., mobile device) 14 for communicating with the nutrition and dietary management platform and providing an interface upon which the user can interact so as to obtain personalized nutritional information and recommendations via the nutrition and dietary management platform.

The mobile device 14 generally includes a computing system 300. As shown, the computing system 300 includes one or more processors, such as processor 302. Processor 302 is operably connected to communication infrastructure 304 (e.g., a communications bus, cross-over bar, or network). The processor 302 may be embodied as any type of processor capable of performing the functions described herein. For example, the processor may be embodied as a single or multi-core processor(s), digital signal processor, microcontroller, or other processor or processing/controlling circuit.

The computing system 300 further includes a display interface 306 that forwards graphics, text, sounds, and other data from communication infrastructure 304 (or from a frame buffer not shown) for display on display unit 308. The computing system further includes input devices 310. The input devices 310 may include one or more devices for interacting with the mobile device 14, such as a keypad, microphone, camera, as well as other input components, including motion sensors, and the like. In one embodiment, the display unit 308 may include a touch-sensitive display (also known as “touch screens” or “touchscreens”), in addition to, or as an alternative to, physical push-button keyboard or the like. The touch screen may generally display graphics and text, as well as provides a user interface (e.g., but not limited to graphical user interface (GUI)) through which a user may interact with the mobile device 14, such as accessing and interacting with applications executed on the device 14, including an app for providing direct user input with asset management services offered by the asset management platform.

The computing system 300 further includes main memory 312, such as random access memory (RAM), and may also include secondary memory 314. The main memory 312 and secondary memory 314 may be embodied as any type of device or devices configured for short-term or long-term storage of data such as, for example, memory devices and circuits, memory cards, hard disk drives, solid-state drives, or other data storage devices. Similarly, the memory 312, 314 may be embodied as any type of volatile or non-volatile memory or data storage capable of performing the functions described herein.

In the illustrative embodiment, the mobile device 14 may maintain one or more application programs, databases, media and/or other information in the main and/or secondary memory 312, 314. The secondary memory 314 may include, for example, a hard disk drive 316 and/or removable storage drive 318, representing a floppy disk drive, a magnetic tape drive, an optical disk drive, etc. Removable storage drive 318 reads from and/or writes to removable storage unit 320 in any known manner. The removable storage unit 320 may represents a floppy disk, magnetic tape, optical disk, etc. which is read by and written to by removable storage drive 318. As will be appreciated, removable storage unit 320 includes a computer usable storage medium having stored therein computer software and/or data.

In alternative embodiments, the secondary memory 314 may include other similar devices for allowing computer programs or other instructions to be loaded into the computing system 300. Such devices may include, for example, a removable storage unit 324 and interface 322. Examples of such may include a program cartridge and cartridge interface (such as that found in video game devices), a removable memory chip (such as an erasable programmable read only memory (EPROM), or programmable read only memory (PROM)) and associated socket, and other removable storage units 324 and interfaces 322, which allow software and data to be transferred from removable storage unit 324 to the computing system 300.

The computing system 300 further includes one or more application programs 326 directly stored thereon. The application program(s) 326 may include any number of different software application programs, each configured to execute a specific task.

The computing system 300 further includes a communications interface 328. The communications interface 328 may be embodied as any communication circuit, device, or collection thereof, capable of enabling communications between the mobile device 14 external devices (e.g., the cloud-based service 12, including the nutrition and dietary management platform 100). The communications interface 328 may be configured to use any one or more communication technology and associated protocols, as described above, to effect such communication. For example, the communications interface 328 may be configured to communicate and exchange data with the nutrition and dietary management platform 100 via a wireless transmission protocol including, but not limited to, Bluetooth communication, infrared communication, near field communication (NFC), radio-frequency identification (RFID) communication, cellular network communication, the most recently published versions of IEEE 802.11 transmission protocol standards, and a combination thereof. Examples of communications interface 328 may include a modem, a network interface (such as an Ethernet card), a communications port, a Personal Computer Memory Card International Association (PCMCIA) slot and card, wireless communication circuitry, etc.

Computer programs (also referred to as computer control logic) may be stored in main memory 312 and/or secondary memory 314 or a local database on the mobile device 14. Computer programs may also be received via communications interface 328. Such computer programs, when executed, enable the computing system 300 to perform the features of the present invention, as discussed herein. In particular, the computer programs, including application programs 326, when executed, enable processor 302 to perform the features of the present invention. Accordingly, such computer programs represent controllers of computer system 300.

In one embodiment where the invention is implemented using software, the software may be stored in a computer program product and loaded into the computing system 300 using removable storage drive 318, hard drive 316 or communications interface 328. The control logic (software), when executed by processor 302, causes processor 302 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software.

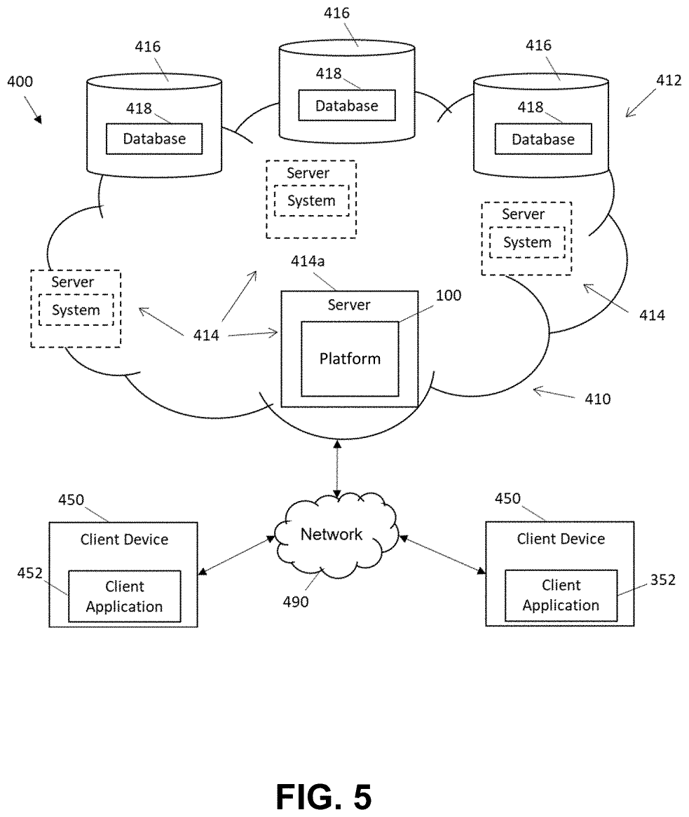

FIG. 5 is a block diagram illustrating an exemplary computing environment for implementing the nutrition and dietary management platform consistent with the present disclosure. As shown above, the environment 400 can include distributed computing system 410 including shared computer resources 412, such as servers 414 and (durable) data storage devices 416, which can be operatively coupled to each other. For example, two or more of the shared computer resources 412 can be directly connected to each other or can be connected to each other through one or more other network devices, such as switches, routers, hubs, and the like. Each of the servers 414 can include at least one processing device (e.g., a central processing unit, a graphical processing unit, etc.) and each of the data storage devices 416 can include non-volatile memory for storing databases 418.

The databases 418 can store data including, for example, data associated ingredients, food items, recipes, prepared foods, and associated attributes and tags, user information and attributes, food graphs, user graphs, diet information and attributes, training data for training artificial intelligence algorithms and machine learning models, test/validation data for testing trained artificial intelligence and machine learning models, parameters and/or coefficients for trained machine learning models, outputs of machine learning models, and/or any other data that can be used for implementing embodiments of the platform 100. An example of the servers 414 is described above with reference to the server 200.

Any one of the servers 414 can implement instances of a platform 100 and/or the components thereof. In some embodiments, one or more of the servers 414 can be a dedicated computer resource for implementing the platform 100 and/or components thereof. In some embodiments, one or more of the servers 414 can be dynamically grouped to collectively implement embodiments of the platform 100 and/or components thereof. In some embodiments, one or more servers 414 can dynamically implement different instances of the platform 100 and/or components thereof.

The distributed computing system 410 can facilitate a multi-user, multi-tenant environment that can be accessed concurrently and/or asynchronously by client devices 450. For example, the client devices 450 can be operatively coupled to one or more of the servers 414 and/or the data storage devices 416 via a communication network 490, which can be the Internet, a wide area network (WAN), local area network (LAN), and/or other suitable communication network. The client devices 450 can execute client-side applications 452 to access the distributed computing system 410 via the communications network 490. The client-side application(s) 452 can include, for example, a web browser and/or a specific application for accessing and interacting with the platform 100. In some embodiments, the client side application(s) 452 can be a component of the platform 100 that is downloaded and installed on the client devices (e.g., an application or a mobile application). In some embodiments, a web application can be accessed via a web browser. In some embodiments, the platform 100 can utilize one or more application-program interfaces (APIs) to interface with the client applications or web applications. In some embodiments, the platform 100 can include an add-on or plugin that can be installed and/or integrated with the client-side or web or mobile applications. Some non-limiting examples of client-side or web applications can include but are not limited to DoorDash, UberEats, GrubHub, restaurant websites or mobile application, and the like. In some embodiments, the platform 100 can provide a dedicate client-side application that can facilitate a communication session between multiple client devices as well as to facilitate communication with the servers 414. An example of the client devices 450 is described above with reference to the computing device 300.

In exemplary embodiments, the client devices 450 can initiate communication with the distributed computing system 410 via the client-side applications 452 to establish communication sessions with the distributed computing system 410 that allows each of the client devices 450 to utilize the platform 100, as described herein. For example, in response to the client device 450a accessing the distributed computing system 410, the server 414a can launch an instance of the platform 100. In embodiments which utilize multi-tenancy, if an instance of the platform 100 has already been launched, the instance of the platform 100 can process multiple users simultaneously. The server 414a can execute instances of each of the components of the platform 100 according to embodiments described herein. The platform 100 executed by the servers 414 can receive text and/or image data from users, which can be used by platform 100 to generate user graphs, map user graphs to the food graph, generate recommendation regarding foods to avoid, foods, to eat, diets to follow, etc.

The system of the present invention further includes an application programming interface (API) that allow business partners, such as food manufacturers or the like, to enter a food formula (i.e. a list of ingredients associated to their quantities) and obtain insights about the formula (i.e. a list of FDA/USDA/EU claims that complies with formula and claims that don't comply with the formula and the reasons for both). Such capabilities is particularly valuable in that, instead of requiring time consuming communications between an R&D team (team that develops a given formula) and any given regulatory entity (entity that greenlights what specific claims are valid for a formula), the scientists can directly have the answer via the system's API at a high confidence threshold, thereby improving the food design process. The API of the system of the present invention is configured to provide food normalization/standardization (i.e., it is configured to address food synonyms, typos, strange input quantity units, etc.) and provide food analysis (i.e., claim and insight retrieval).

As previously described herein, the nutrition and dietary management platform 100 is configured to provide personalized nutritional information and recommendations to a user based, at least in part, on artificial intelligence (AI) and big data analytics.

For example, such personalized nutritional information and recommendations are based on the processing sets of data, including a food graph and a user graph, which are generated using a data hierarchy structure leveraging food attributes (i.e., nutrition facts, food tags, etc.) and user attributes (i.e., the user attributes previously described herein), respectively. The food graph may generally include a set of data associated with various food items, such as ingredients, products, recipes, and the like, which may be obtained from a database that has been populated with millions of food items and associated information. Such food items and associated information is generally acquired from various data sources (including publicly available data sources for any given food item). The user graph may generally include a set of data associated with the user attributes. Upon generating food and user graphs, the system is able to programmatically build mappings between the food graph and the user graph using AI-based techniques, such as machine learning, natural language processing, reinforcement learning, and data mining techniques. Upon establishing mappings between the food graph and user graph, the system is configured to autonomously generate one or more dietary predictions, dietary recommendations, and/or dietary modifications personalized to a given user based on their attributes and preferences.

For example, the platform 100 is able to programmatically find mappings between the food graph and the user graph using via the graph mapping engine 110 and/or AI engine 112 of the platform 100.

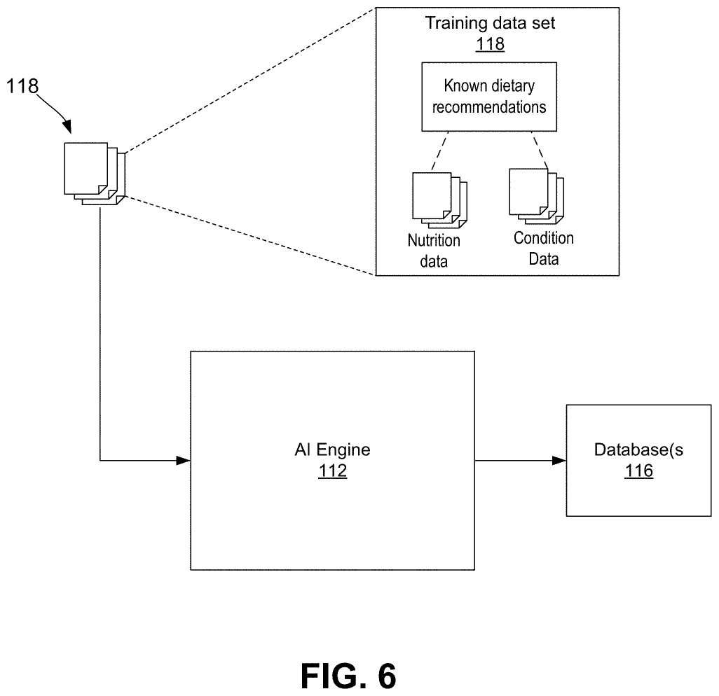

For example, the AI engine 112 may be configured to run a neural network that has been trained using a plurality of training data sets that include qualified reference data. FIG. 6 is a block diagram illustrating inputting of reference data (i.e., training data sets) into the AI engine 112, for example. The machine learning techniques of the present invention, and the subsequent analysis of food graphs and user graphs based on such techniques may utilize reference data. The reference data may include a plurality of training data sets 118 inputted to the AI engine 112 of the present invention. For example, each training data set includes reference dietary data, which may include, for example, known dietary recommendations including known nutrition data of any food item and known condition data associated with the known nutrition data of a given food item. The condition data may include, for example, known dietary restrictions and/or conditions associated with any given food item.

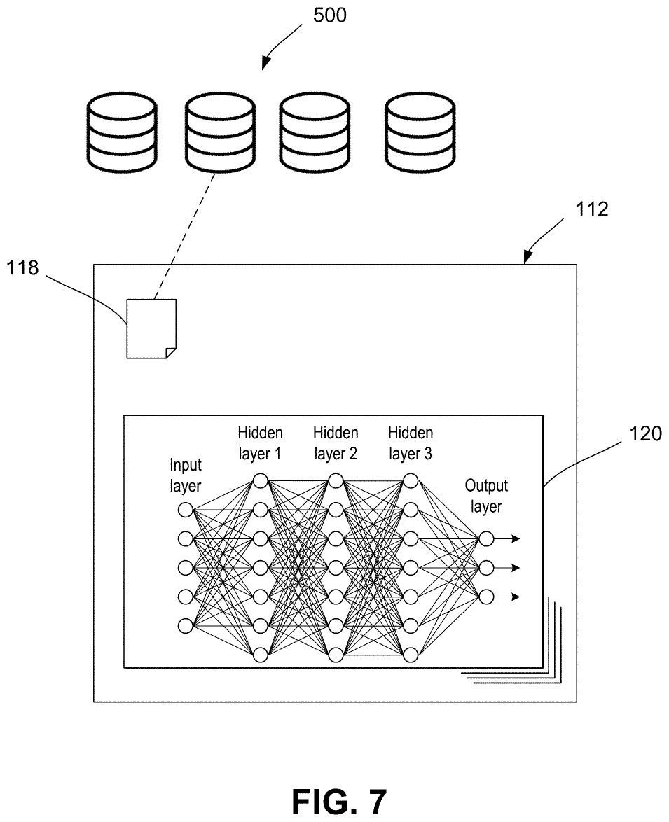

FIG. 7 shows an exemplary AI engine 112 according to certain embodiments of the present disclosure. The AI engine 112 may access reference data from the one or more training data sets 118 provided by any known source 500. The source 500 may include, for example, laboratory-specific repository of reference data collected for purposes of machine learning training. Additionally, or alternatively, the source 500 may include publicly available registries and databases and/or subscription-based data sources. For example, data may be made available from food manufacturers and/or food-related federal agencies, including, but not limited to, the United States Food and Drug Administration (FDA), the United States Department of Agriculture (USDA), Centers for Disease Control and Prevention (CDC), European Food Safety Authority (EFSA), and the like.

The plurality of training data sets 118 may generally feed into the AI engine 112. The AI engine 112 may include, but is not limited to, a neural network, a random forest, a support vector machine, a Bayesian classifier, a Hidden Markov model, an independent component analysis method, and a clustering method.

For example, the AI engine 112 may include an autonomous machine learning system that associates the condition data with the reference nutrition/food data. For example, the AI engine may include a deep learning neural network that includes an input layer, a plurality of hidden layers, and an output layer. The autonomous machine learning system may represent the training data set using a plurality of features, wherein each feature comprises a feature vector.

For example, the autonomous machine learning system may include a convolutional neural network (CNN). In the depicted embodiment, the AI engine 112 includes a neural network 120.

The AI engine 112 discovers associations in data from the training data sets. In particular, the AI engine 112 processes and associates the reference nutrition/food data and condition data with one another, thereby establishing reference data in which reference dietary recommendations/guidelines are established. The reference data is stored within a database 116, for example, and available during subsequent processing of food graph and user graph data to thereby provide personalized nutritional information and recommendations to a user.

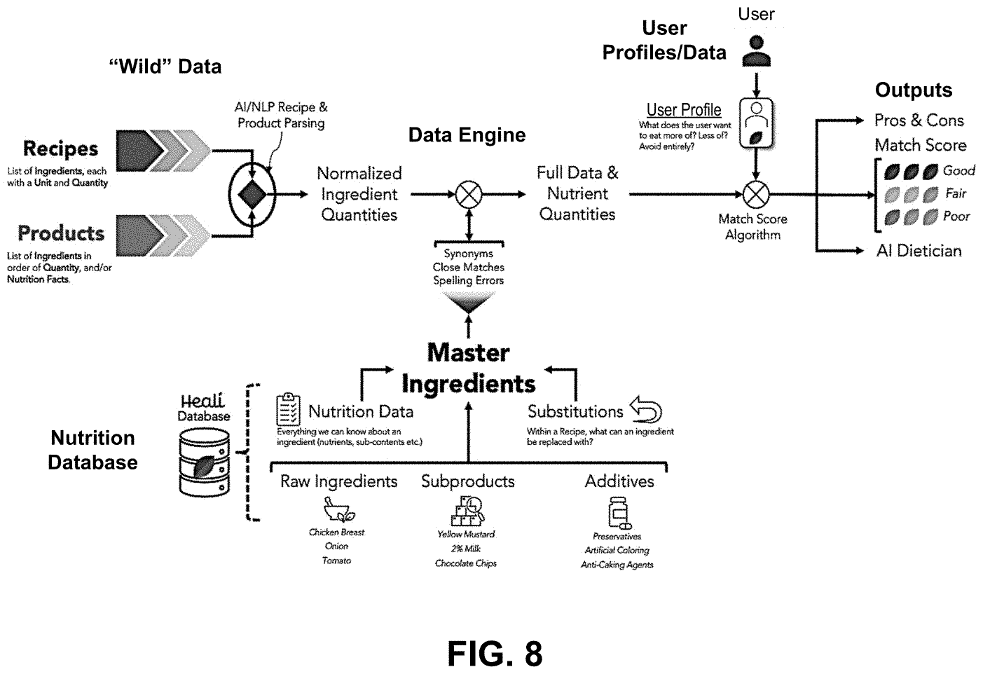

FIG. 8 is a flow diagram illustrating the overall process for providing nutritional and dietary guidance services and solving precision nutrition problems via the nutrition and dietary management platform consistent with the present disclosure.

As previously described herein, the nutrition and dietary management platform 100 provides a suite of features that allows for a user to obtain personalized nutritional information and recommendations based on artificial intelligence (AI) and big data analytics.

In particular, a user may initially create profile in which they provide basic information (i.e., name, age, gender, contact information, etc.). The platform further allows for a user to provide other attributes, including, but not limited to, biometric data (i.e., DNA and/or user microbiome data), demographic data, user food and/or symptom logs, user medical conditions, user lab results, etc.). Additionally, the platform allows for a user to set certain preferences, such as a preferred dietary lifestyle (i.e., vegan, vegetarian, paleo, ketogenic, gluten free, dairy free, Mediterranean, etc.), as well as preferred nutrient intake/avoidance, such as less of a specific nutrient or vitamin (e.g., less calories, less carbohydrates, less fat, less sugar, less sodium, less cholesterol, etc.) or more of a specific nutrient or vitamin (e.g., more protein, more fiber, more iron, more calcium, etc.).

Upon a user establishing their attributes and preferences, the nutritional guidance system of the present invention is able to determine optimal eating plan for the user. More specifically, the nutrition and dietary management platform is configured to provide personalized nutritional information and recommendations to a user based, at least in part, on artificial intelligence (AI) and big data analytics. For example, such personalized nutritional information and recommendations are based on the processing sets of data, including a food graph and a user graph, which are generated using a data hierarchy structure leveraging food attributes (i.e., nutrition facts, food tags, etc.) and user attributes (i.e., the user attributes previously described herein), respectively. The food graph may generally include a set of data associated with various food items, such as ingredients, products, recipes, and the like, which may be obtained from a database that has been populated with millions of food items and associated information. Such food items and associated information is generally acquired from various data sources (including publicly available data sources for any given food item). The user graph may generally include a set of data associated with the user attributes. Upon generating food and user graphs, the system is able to programmatically build mappings between the food graph and the user graph using AI-based techniques, such as machine learning, natural language processing, reinforcement learning, and data mining techniques. Upon establishing mappings between the food graph and user graph, the system is configured to autonomously generate one or more dietary predictions, dietary recommendations, and/or dietary modifications personalized to a given user based on their attributes and preferences.

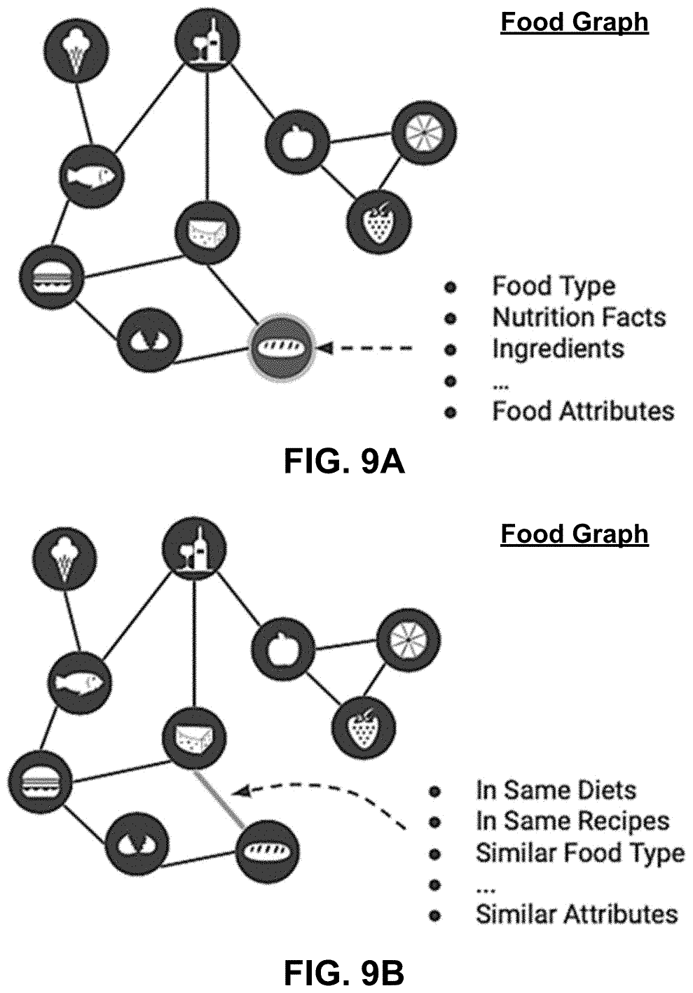

FIGS. 9A and 9B are exemplary depictions of a food graph generated via a graph generator module consistent with the present disclosure, wherein the food graph nodes represent food items (see FIG. 9A) and a relationship between food items is captured on edges of the food graph (see FIG. 9B). A food graph can be generated by a graph generator module 106 of the platform 100. The food graph nodes represent food items such as ingredients, products, recipes, etc, and have attributes such as nutrition facts, food tags, and the like. The relationship between food items is captured on the edges of the graph. Edges encode how similar food items are in term of their attributes.

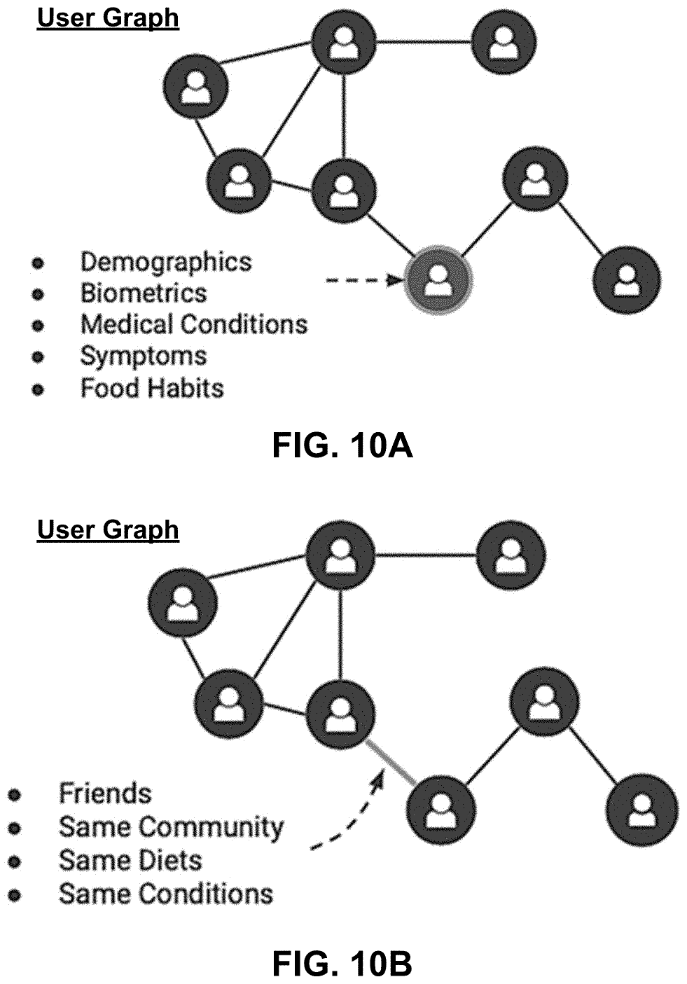

FIGS. 10A and 10B are exemplary depictions of a user graph generated via a graph generator module consistent with the present disclosure, wherein the user graph nodes represent users with attributes (see FIG. 10A) and a relationship between attributes is captured on edges of the user graph (see FIG. 10B). User graphs can be generated by the graph generator module 106 of the platform 100. Similar to the food graph, the user graph nodes represent users with attributes such as biometrics and demographics data, food and symptom logs, medical conditions, lab results, DNA data, and the like. Edges capture user correlation in terms of their attributes such as similar demographics and biometrics, similar diet, similar community, similar symptoms or medical conditions, and the like.

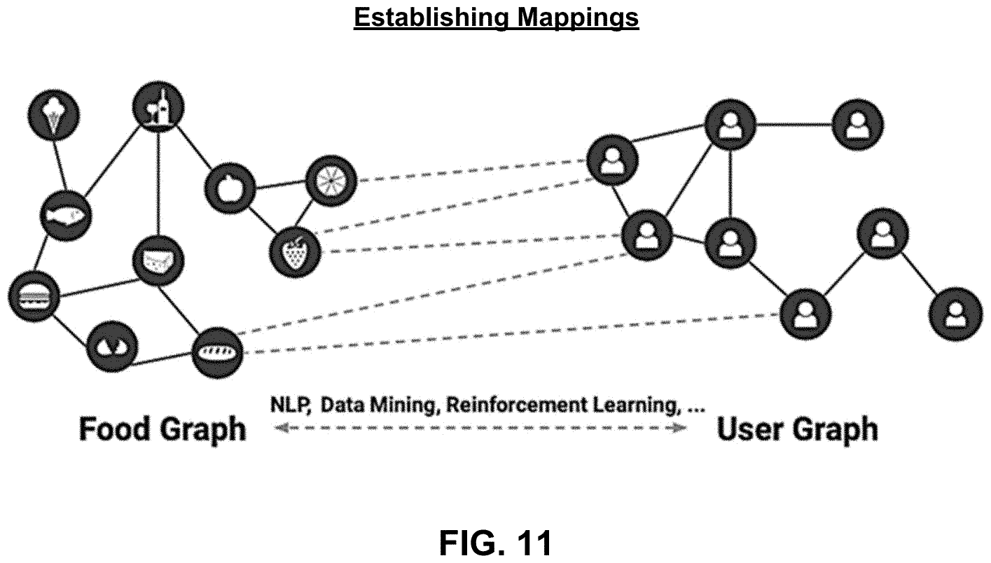

In order to solve precision nutrition problems, embodiments of the present can programmatically find mappings between the food graph and the user graph using a graph mapping engine 110 and/or an AI engine 112 of the platform 100. For example, using such techniques, including, but not limited to, machine learning, natural language processing, reinforcement learning, and data mining techniques, the systems and methods of the present invention can autonomously solve problems such as personalized nutrition and meal planning automation. For example, FIG. 11 is an exemplary depiction of mappings established between the food graph and the user graph based on AI-based techniques, including, but not limited to, machine learning, natural language processing, reinforcement learning, and data mining techniques.

As previously described herein, there is a data problem associated with the understanding of nutritional characteristics of ingredients, prepared foods, food products, and recipes. Such a problem exists because there is missing and/or hidden data that is required for determining and fully understanding the nutritional characteristics of ingredients, prepared foods, food products, and recipes.

For example, with regard to the food graph aspects, the USDA and FDA impose food manufacturers, food retailers, and restaurants with a certain number of locations (i.e., 20 or more locations under the same entity name) to disclose their product information. However, such data can be highly unstructured, may include typographical errors, incorrect data values, missing data, and even hidden data. This is partly due to the fact that the USDA and the FDA typically impose a loose and restricted set of requirements to food manufacturers and food retailers, and further lack adequate bandwidth to control the flow of new products or product changes every year. For example, in the case of a maple syrup, seeming identical maple syrup products from two different manufacturers may have completely different nutrition data provided on their labels (i.e., different suggested service sizes, unnormalized quantities, etc.). Accordingly, as a result of such relaxed requirements, food labels can be counterintuitive. For example, a consumer may be led to believe that some fruits are worse in term of sugar when compared to maple syrup or that other products are worse because they look more processed (i.e., added sugar content appears higher than that of maple syrup, even though that is not the case. In summary, nutrition facts are misleading because the USDA and FDA requirements are too loose and difficult to check as a result of the massive scale of new products being introduced into the market every year. Furthermore, only 15 nutrients are required to appear on labels, including calories, total fat, saturated fat, trans fat, cholesterol, sodium, total carbohydrates, dietary fiber, total sugars, added sugars, protein, vitamin D, calcium, iron, and potassium.

As a result, such food data-related challenges are extremely confusing for the consumer. The systems and methods of the present invention solve such issues by: 1) better clarifying the definition of food items; 2) adding thousands of food tags for each food item; 3) developing custom nutrient and custom insights and normalizing daily value (DV); and 4) unravelling the quantity of each ingredients in a product, as well as unraveling hidden nutrients.

It should further be noted that the consumer side also has its share of challenges. For example, user-related data is difficult to collect in an accurate and consistent manner (e.g., conventional systems require that users enter their data at every meal and without errors). The systems and methods of the present invention address such challenges at a user interface and user interaction level by providing a simple interface that maximizes the precision of the data entered, while minimizing the friction of entering the data, thereby reducing the chance of inaccuracies in data entry.

The following description includes acronyms, terminologies, and definitions of some of the nutrition concepts, particularly with regard to food graph technology and food graph-user graph mapping technology. Such acronyms, terminologies, and definitions are provided herein.

### Reference Amount Customarily Consumed (RACC)

Usually, on the food label, the RACC is listed as servings per container and serving size. The FDA provides guidelines for most types of food on how to report serving sizes on food labels. Food manufacturers can put from 50% up to 200% of the recommended serving. For example if the FDA recommends putting 60 g as a serving size for maple syrup, food manufacturers can report from 30 g to 120 g as serving size on the back of their maple syrup product. Because nutrients are reported per serving size, and maple syrup contains a lot of sugar, typically food manufacturers will pick 30 g as serving sizes, so that the amount of sugar does not look too high on the label.

### Recommended Daily Intake (RDI)

The RDI is the estimated amount in grams or milliliters of a nutrient recommended per day, defined by the FDA. For example the FDA recommends no more than 50 g of sugar per day, hence rdi of sugar =50 g.

### Daily Value (DV)

The DV represents the % of nutrient per serving (RACC) with respect to its RDI. For example if a product contains lOg of sugar per serving, and knowing that the sugar RDI is 50 g, the sugar DV is 10/50=20%. Daily values are posted next to the amount on food labels. The lower the serving size, the lower the DV, because nutrients amount on food labels are listed per serving size.

### Medical Nutrition Therapy (MNT)

Medical nutrition therapy (MNT) is a key component of diabetes education and management. MNT is defined as a nutrition-based treatment provided by a registered dietitian nutritionist. It includes a nutrition diagnosis as well as therapeutic and counseling services to help manage medical conditions such as diabetes. In addition to users demographic and biometric data, when a user knows their medical conditions, the systems and methods of the present invention use MNT research to seed users diet when they enter their food preferences via the interface.

### Food Graph Concepts and Technologies

### Food Items:

The platform 100 defines five types of food items: 1) ingredients (unprocessed raw food); 2) additives: 3) sub-products (ingredients made of other ingredients); 4) products (ingredients or sub-products that can be purchased and contain a list of ingredients, even if the ingredient is the product itself); and 5) recipes. Furthermore, the platform 100 defines a “broad” food item as a macro/generic food item with no nutrition facts, like “fruit” or “spice”. Those food items appear in product and recipe sometimes and the final match score will only consider tags associated with those ingredients, since they are lacking any nutrition info. The platform 100 also defines the notion of synonym (chickpea is a synonym of garbanzo bean) an ingredient that is identical to another, except for its name. The platform 100 defines “close match” ingredients as ingredients for which the platform 100 could not find nutrition information, but where the platform 100 finds similar ingredients with nutrition information that can be used as a proxy.

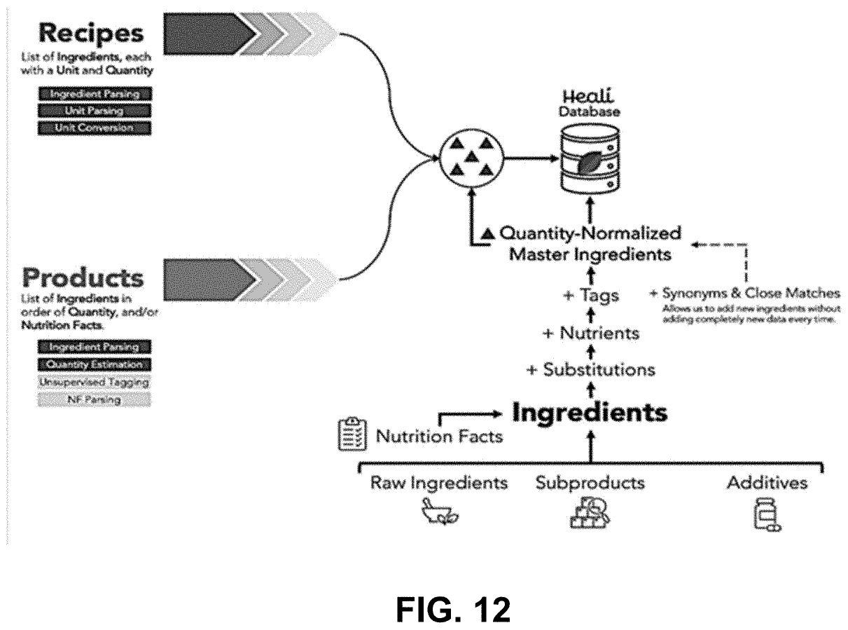

FIG. 12 is a flow diagram illustrating the overall process for populating a database with food items and organizing/categorizing various aspects of a given food item via the nutrition and dietary management platform consistent with the present disclosure.

### Food Tags:

The platform 100 has defined thousands of food tags for each ingredient, additives and sub-product. Food tags represent a specific food attribute such as FRUIT, VEGETABLE, LEGUME, GRAIN, and ANIMAL_PRODUCT, and are organized under a hierarchy. For example, MEAT, DAIRY and SEAFOOD are under ANIMAL_PRODUCT, GAME_MEAT and POULTRY are children of MEAT, CHICKEN a child of POULTRY, and so on.

The database of the platform 100 comprises millions of products and recipes (rows), combined with the food tags for each item (column), and allows for billions of database entries. It should be noted that it is impractical to perform data augmentation, annotation and correction manually in a realistic time frame on such a large number of entries. To that extent, tags are programmatically propagated to products and recipes by a tagging engine based on the list of ingredients and their quantities. The propagation works as follows:

Given the list of ingredients/sub-products in a food item, and their respective tag list, the tagging engine 108 of the platform 100 concatenates the tag list of each of those ingredients/sub-products by keeping the max probability of each tag. For example, for a soup made of carrots, basil and broth, each ingredient/sub-products have the following tags:
Carrot: VEGETABLE: 1
Basil: HERB: 1
Broth: VEGETABLE: 1, HERB: 0.5, ANIMAL PRODUCT: 0.5
   Where VEGETABLE: 1 means tag VEGETABLE with probability 1. The soup will have the tag list:

VEGETABLE: max(1, 1), HERB: max(1, 0.5), ANIMAL_PRODUCT: max(0.5)=VEGETABLE:1, HERB: 1, ANIMAL PRODUCT: 0.5

Tags are shown in the app when they are relevant to explain a match status in a pros/cons section of the user interface.

### Custom Nutrient/Insight:

Custom nutrients (also referred as custom insights) are special food metrics designed to work similarly to nutrients on the back of a product food label. The platform 100 includes several custom nutrients, as described below.

Plant content: estimated % of plants (fruits, vegetables, grain, etc) in a food item. Binary for ingredients and additives (0 or 100%), manually estimated for sub-products, depends on the list of ingredients+their quantities for recipes. For products, it's a non-linear combination of the first k ingredients plant content, where k is estimated from the product information context (such as the number of ingredients, the product type, etc).

Protein quality: evaluate the quality protein content with respect to the total saturated fat. For example the protein quality coming from 100 g of chicken wings is higher than from pork belly because chicken wings contain less saturated fat. The platform 100 defines protein quality as a nonlinear combination of nutrient (such as protein and saturated fat) amounts.

Sugar Impact: evaluate the impact of sugar with respect to fiber and protein content. The idea is that getting your source of sugar from fruits and some dairy products is not as bad as getting it from high fructose corn syrup. The problem is that manufacturers, following FDA guidelines for serving sizes (i.e. RACC), tends to put small serving sizes for products containing high amounts of sugar. So you can have a product with a high concentration of processed sugar with a small serving size and still get a reasonable sugar DV. In addition, products that are almost pure sugar, even processed, have their sugar amount reported as natural sugar instead of added sugar. To counteract manufacturers exploiting loopholes in the FDA guidelines, the platform 100 measures the sugar impact as a non-linear combination of nutrient (such as sugar, added sugar, fiber and protein) amounts.

Net Carb for Keto: evaluate the amount of net carbs, adjusted for the ketogenic diet. Defined as a non-linear combination of nutrient (such as carbohydrates, fiber and sugar alcohols) amounts.

Calories from carbs: evaluate the percentage of calorie from carbohydrates as a non-linear combination of nutrient (such as carbohydrates and calories) amounts.

Fat for Keto: adjusted amount of fat for the keto diet as a non-linear combination of nutrient (such as fat) amounts.

### Nutrient DV Adjustment

If a diet limits the amount of recommended sugar, then products that contain sugar and have a high serving size, like juices or large fruits, will be more penalized than concentrated, extract and small container products with much smaller serving sizes, like small corn syrup bottles and cookies. To this extent, the AI engine 112 of the platform 100 adjusts all the data values (DVs) before using them in any equations so that extreme (both and high) serving sizes have a dampened effect on the food analysis.

### Product Ingredients Quantity Estimation

Product food labels list the ingredients contained in each product, ordered by their amount (first ingredient has the highest amount). However labels do not specify the amount of each ingredient per say. The platform 100 can derive multiple solutions to infer the ingredient amounts. It should be noted that that while embodiments of the systems and methods of the present invention are geared towards unraveling hidden data behind product ingredient quantities, the same techniques can be applied to any domain where the AI engine 112 of the platform 100 can formulate the problem as a linear least square problem with coefficient ordering preservation constraints:

x*=min∥Ax−y∥²s.t. x₀>x₁>x₂ . . . >xₙ and Sum xᵢ=1

Where x is the unknown vector to optimize and A represents the data matrix, each column being a set of features of a data point, and y the output/target of the linear combination of each column of A.

### Quadratic (QP) Optimization Based:

The platform 100 can have access to the final product nutrients, and can collect nutrients for over 30,000 additives, ingredients and sub-products. The AI engine 112 of the platform 100 formulates the quadratic problem as follows:

x*=min∥Ax−y∥²s.t. x₀>x₁>x₂ . . . >xₙ and Sum xᵢ=1

Where x is the vector representing the percentage of each ingredient in the product, A is a matrix with column i representing the nutrient amounts for the ith ingredient in the list and y represents the product nutrient amounts. Only the nutrients common to all the ingredients and the product are used. ∥ ∥ is the Euclidean norm.

In addition the AI engine 112 of the platform 100 measures the final fit error as:

err=∥Ax*−y∥²

The AI engine 112 of the platform 100 can use replicator dynamics to solve the QP and estimate x*, see paper “Fast Population Game Dynamics for Dominant Sets and Other Quadratic Optimization Problems” from Samuel Rota Bulò, Immanuel M. Bomze, and Marcello Pelillo. They proposed a fast population game dynamics, motivated by the analogy with infection and immunization processes within a population of “players” for finding dominant sets, a powerful graph-theoretical notion of a cluster. Each step of the proposed dynamics is shown to have a linear time/space complexity and they show that, under the assumption of symmetric affinities, the average population payoff is strictly increasing along any non-constant trajectory, thereby allowing them to prove that dominant sets are asymptotically stable (i.e., attractive) points for the proposed dynamics. The approach is general and can be applied to a large class of quadratic optimization problems. Experimentally, their dynamics is found to be orders of magnitude faster than and as accurate as standard algorithms.

Order based:

Ingredients are listed in decreasing order based on their amount in the product. The platform 100 can define x the vector representing the percentage of each ingredient in the product using a cubic decay function:

xᵢ=a*i^3+b*i^2+c*i+d

Where the coefficients are regressed using a least square fitting approach with M-estimator for outliers pruning by the AI engine 112 of the platform 100, based on an in-house training set and such that Sum xᵢ=1.

ML Order based:

Similarly to the order based method, the AI engine 112 of the platform 100 can define x the vector representing the percentage of each ingredient in the product using a cubic decay function:

xᵢ=a(ingredients)*i^3+b(ingredients)*i^2+c(ingredients)*i+d(ingredients)

Where the coefficients a, b, c, d are regressor functions based on the ingredient list and trained on an in-house training set with the constraints that x₀>x₁>x₂>xₙ and Sum xᵢ=1.

### Hybrid:

The AI engine 112 of the platform 100 can compute x the vector representing the percentage of each ingredient in the product as:

x=(1−α) x*+αx**

Where x* is computed using the QP method, x** using the ML order based and a is based on the error estimated with the QP method so that a=0 if err=0, α=1 if err is high:

α=1−ln (1+exp(−b*err))/ln(2)

where b is fine-tuned on an in-house training set using a least square fitting approach with M-estimator for outliers pruning by the AI engine 112 of the platform 100.

### Inferred Nutrients and Compounds

The FDA does not require food manufacturers to provide all product nutrients on the product nutrition fact labels. Their quantity, if less than a threshold, can be considered 0 or be rounded to the upper or lower valid integer. See https://www.fda.gov/files/food/published/Food-Labeling-Guide-%28PDF%29.pdf for detailed information.

This is particularly penalizing users on a diet that target nutrients that are not required on food labels by the FDA. For example a user with kidney disease who wants to limit its intake of phosphorus would not know what product to buy and in what quantity to buy based on the food labels, because phosphorus is rarely reported as it is not part of the required list of nutrients imposed by the FDA.

The AI engine 112 of the platform 100 can use a product ingredient quantity estimation engine to recover those hidden nutrients and compounds. For example, if a soup has an ingredient list, such as, for example: water, potato, carrots, onions, and the quantity estimation engine produces: 50% water, 25% potato, 20% carrots, 5% onions. A such, the platform 100 can derive hidden nutrients as follows. First, the platform 100 can convert the quantity percentages into grams by multiplying the percentages with the mass/volume of the product. For a soup can that weighs 400 g, the platform 100 can determine that there is 200 ml of water, 100 g of potatoes, 80 g of carrots and 20 g of onions. Let's assume now the platform 100 is operating to infer the amount of vitamin C in the soup. The water does not contain any, potatoes contains 19.7 mg for 100 g potatoes, hence for 200 g it produces 200*19.7/100 mg, for 80 g of carrots: 80*5.9/100 mg, for 20 g of onion: 20*7.4/100. The total amount Vitamin C in the soup is then 200*19.7/100+80*5.9/100+20*7.4/100=45.6 mg. The general formula executed by the platform 100 is:

inferred nutrient quantity n=q/100*Sum p_i*n_i

Where q is the over mass (g) or volume (ml) of the product, p_i is the percentage of each ingredient in the product, n_i is the amount of a selected nutrient per 100 grams/ml of that the ith ingredient. The platform 100 can apply similar logic for other compounds like FODMAPs.

### Food Graph—User Graph Mapping Concepts and Technologies

### Diet Definition

Each diet is defined with three sets of wish lists: 1) the avoids; 2) the eat mores; and 3) the eat lesses. The avoid list contains the list of nutrients, ingredients and food tags to avoid at all cost. The eat more list contains a list of nutrients, ingredients and tags a user wants to eat more of. The eat less list contains a list of nutrients, ingredients and tags a user wants to eat less of. When multiple diets are merged together, the resulting diet is the most restrictive intersection of all the input diets. Preset diets also contain extra metadata such as name, description, etc.

In addition, diets evolve with time. Based on food and symptom logs, users can refine their own diet. The AI engine 112 of the platform 100 can also provide programmatically diet change recommendations from the user food and symptom logs using Q-learning based reinforcement learning prediction inspired by the paper “Reinforcement Learning: Prediction, Control and Value Function Approximation” from Haoqian Li and Thomas Lau.

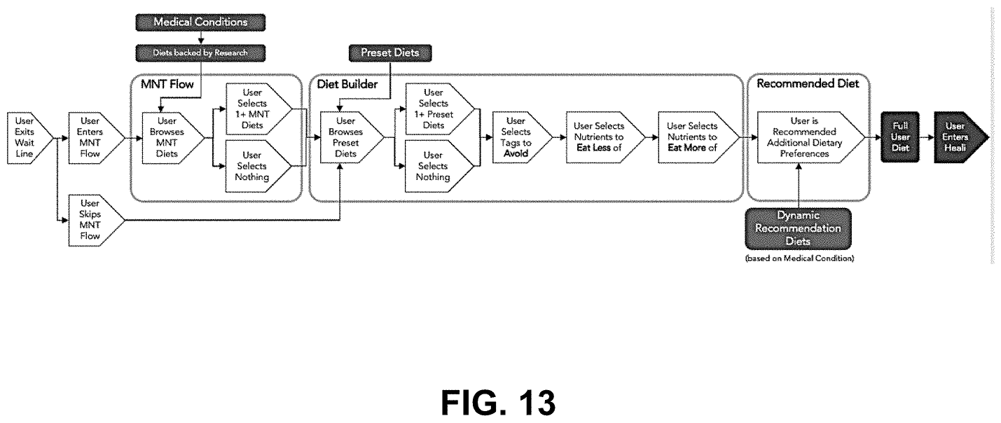

FIG. 13 is a flow diagram illustrating the general process in which a user can establish their dietary preferences via the nutrition and dietary management platform. For example, as previously described herein, a user may first initiate a mobile application on their smartphone in which they may interact with the nutrition and dietary management platform via a GUI. Upon initiating the app, the user is able to select from a suite of features that allows them to obtain personalized nutritional information and recommendations based on artificial intelligence (AI) and big data analytics.

In particular, a user may initially create profile in which they provide basic information (i.e., name, age, gender, contact information, etc.). The platform further allows for a user to provide other attributes, including, but not limited to, biometric data (i.e., DNA and/or user microbiome data), demographic data, user food and/or symptom logs, user medical conditions, user lab results, etc.). Additionally, the platform allows for a user to set certain preferences, such as a preferred dietary lifestyle (i.e., vegan, vegetarian, paleo, ketogenic, gluten free, dairy free, Mediterranean, etc.), as well as preferred nutrient intake/avoidance, such as less of a specific nutrient or vitamin (e.g., less calories, less carbohydrates, less fat, less sugar, less sodium, less cholesterol, etc.) or more of a specific nutrient or vitamin (e.g., more protein, more fiber, more iron, more calcium, etc.).

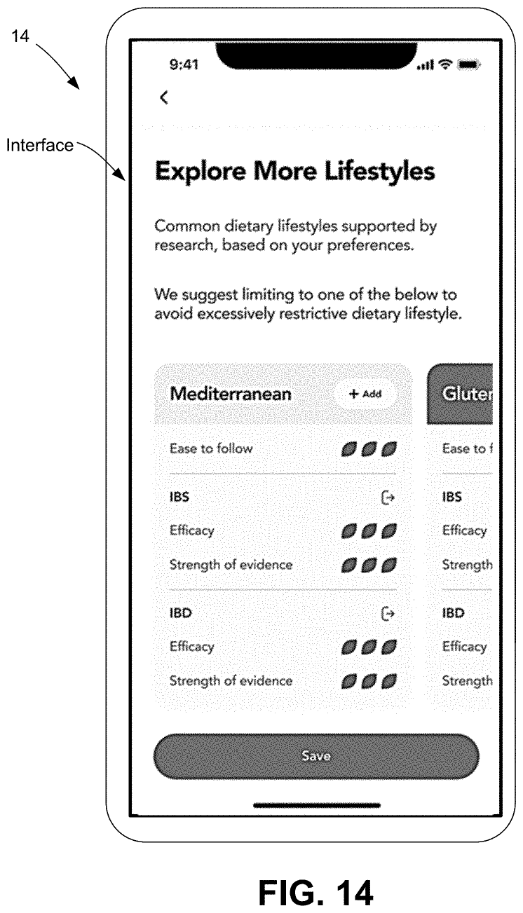

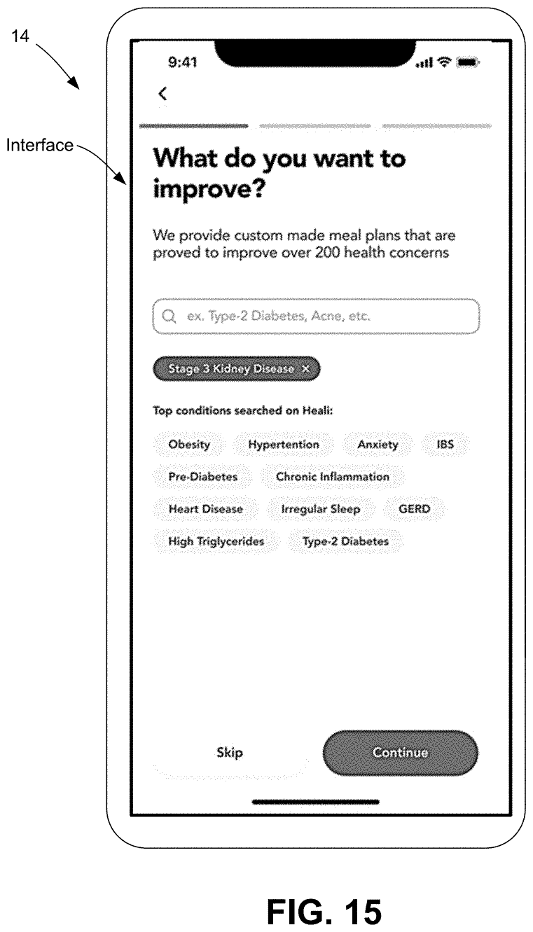

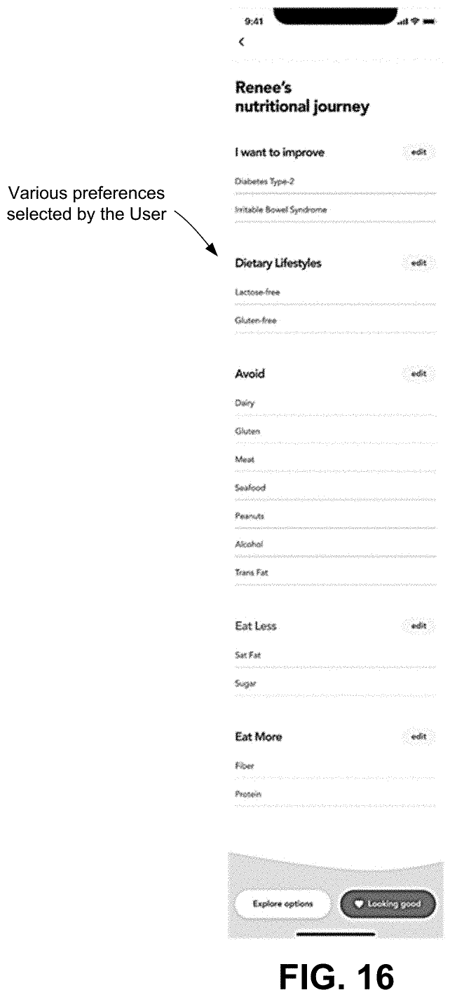

For example, FIGS. 14 and 15 are screenshots of an interface on a mobile device associated with the nutritional and dietary guidance services provided by the nutrition and dietary management platform of the present disclosure, in which a user is navigating through the initial onboarding process, including selected specific dietary needs or desired dietary lifestyle. FIG. 16 is a screenshot of an exemplary interface providing a summary of a user's various preferences with regard to their medical nutrition therapy and/or preferred dietary lifestyle, which further may include preferred nutrient intake and avoidance.

Upon establishing their profile, include selecting their preferences, a user can search for specific food items and/or recipes, and the system is further configured to provide a user with recommended food items, meals, recipes, and the like. For example, in some instances, the platform is configured to automatically provide a user with recommended food items, recipes, meal plans, and the like, based on a user set preference (i.e., the user may set a preference for the platform to automatically generate a daily, weekly, and/or monthly suggested meal plan).

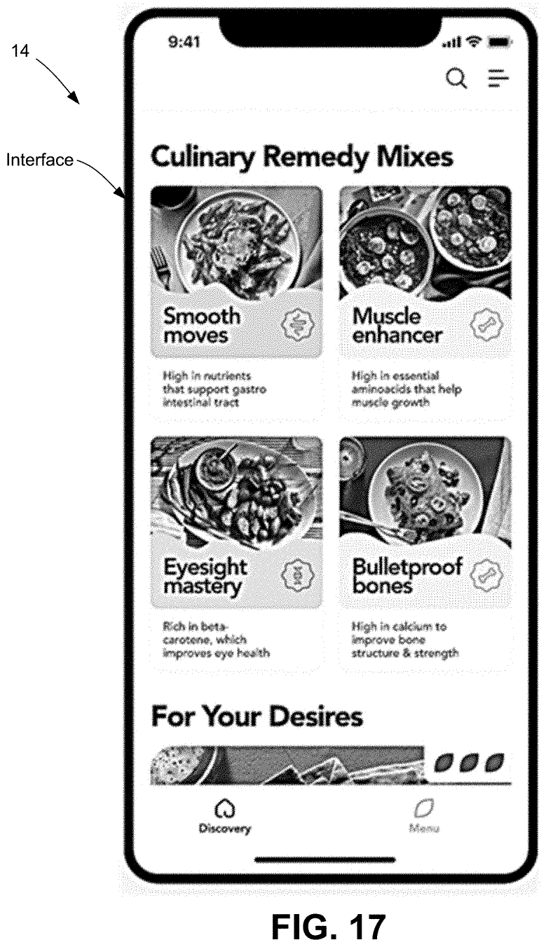

FIG. 17 is a screenshot of an exemplary interface providing the user with different food options that provide specific benefits (i.e., foods and recipes for addressing digestion-related issues, promoting muscle growth, improving bone structure and strength, improving eyesight, and the like).

In some embodiments, a user may directly interact with the platform to find a given food item, recipe, or meal plan. For example, a user may use their smartphone to take a photograph of a menu item or scan a food item barcode.

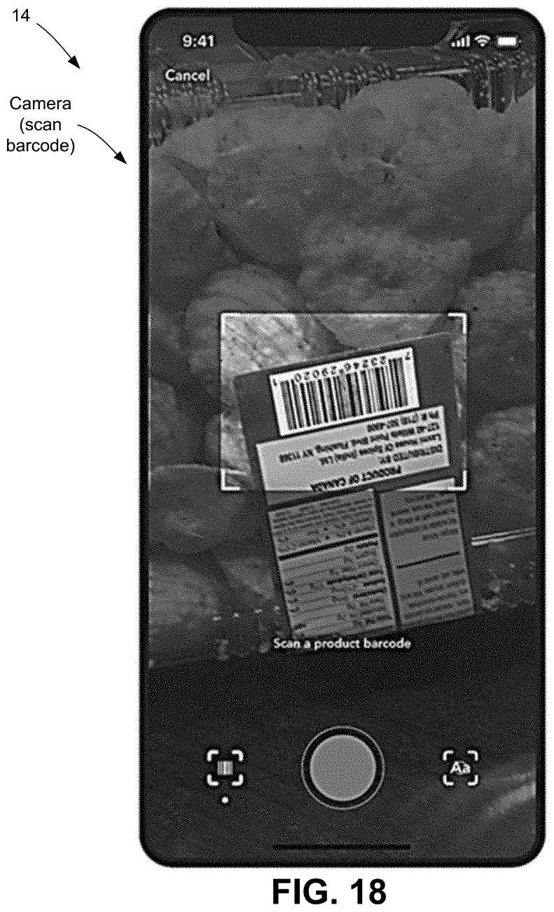

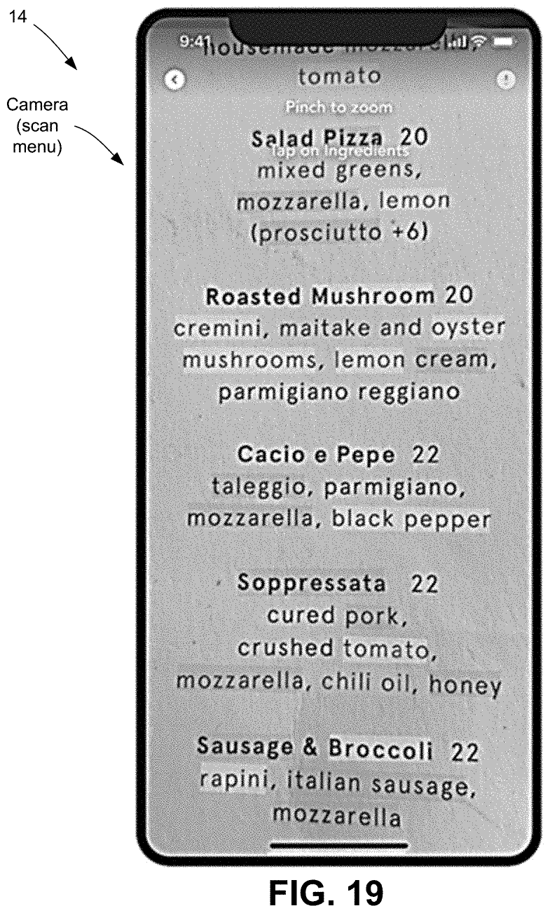

FIGS. 18 and 19 are screenshots illustrating a user utilizing their mobile device to scan a food item barcode (see FIG. 18) or capture an image of a menu item (see FIG. 19), wherein the system of the present disclosure is configured to identify the scanned food item or specific meals on the menu and provide the user with nutritional information associated therewith. For example, the system of the present invention is configured to identify the menu item (and all related food items in that menu item) or the food item and provide the user (via the platform interface) with an indication as to whether such foods adhere to the individualized nutritional preferences and, in the event that such foods do not adhere to the preferences, the system is configured to provide alternative options to choose.

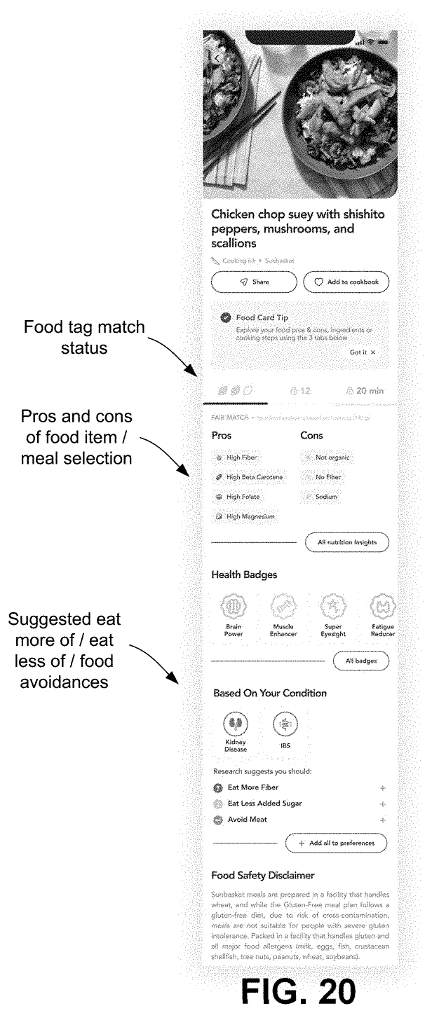

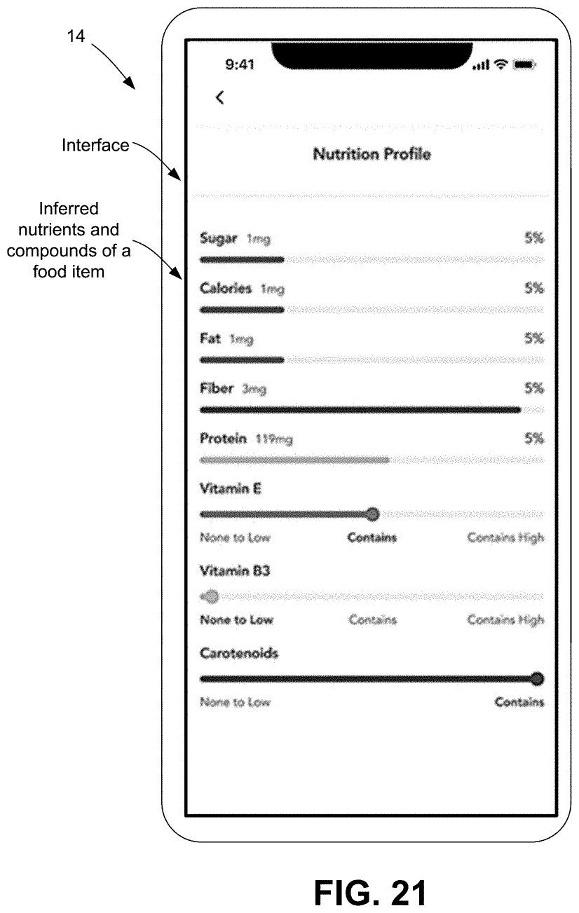

FIG. 20 is a screenshot of an interface on a mobile device associated with the nutritional and dietary guidance services provided by the nutrition and dietary management platform of the present disclosure, in which a user is presented with an indication as to the degree to which a food item or recipe or meal matches a user's personalized nutritional information and recommendations. FIG. 21 is a screenshot of the interface on a user's mobile device, in which a user is presented with a nutrition profile of a given food item of interest.

### Match Score, Match Status

The match score is a score, between 0 and 1, that reflects how well a food item complies to a diet definition. The higher the score, the better the match. The match score is a non-linear combination of the avoid score, the eat more score and the eat less score. The avoid score reflects the likelihood of the presence of certain ingredients, or nutrients to be avoided. Similarly, the eat less score and eat more score represent the likelihood of ingredients and nutrients to impact a user's diet based on the user's preferences (eat more/less of certain ingredients or nutrients). The avoid, eat more, and eat less scores non-linearly combine many variables such as nutrition facts and food tags and food type and reflect FDA recommendation guidelines.

The match status has three possible values: poor, fair and good and is defined as follows:
match status=poor if match score<⅓
match status=fair if ⅓<match score<⅔
match status=good if match score>⅔

Only the match status is visible for users. When the match status is poor the platform 100 can display one red leaf followed by two gray leaves, when the status is fair, the platform 100 can display two yellow leaves followed by one gray leaf, when the score is good, the platform 100 can display three green leaves. Sometimes the platform 100 can refer the color red, yellow, green to their corresponding status poor, fair and good, like when a food item is red, it means that the match status is poor.

### Messaging

The match status for a food item often comes back as fair (yellow). A fair score can be ambiguous for users because the action item they should take depends on their interpretation of what's fair. In contrast, when a food item has a poor or good status, it's clear for the user what to do. To that extent, when a match status is fair, the user interface of the platform 100 can give to our user additional explanation that they can use to make a judgment call about the food item.

There are many reasons why a match status is fair, in the following the platform 100 can define the seven most important ones that the user interface of the platform 100 explicitly conveys to users in the form of an “ingredient alert” pop up messaging.

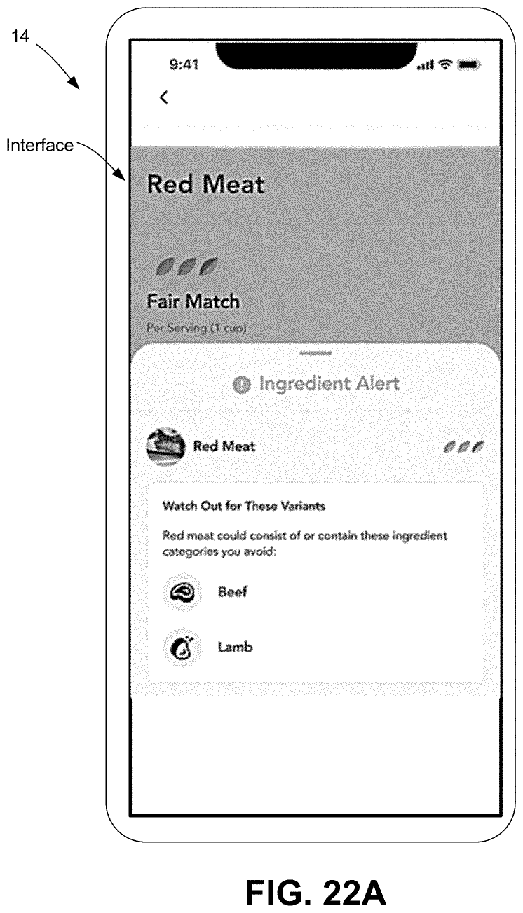

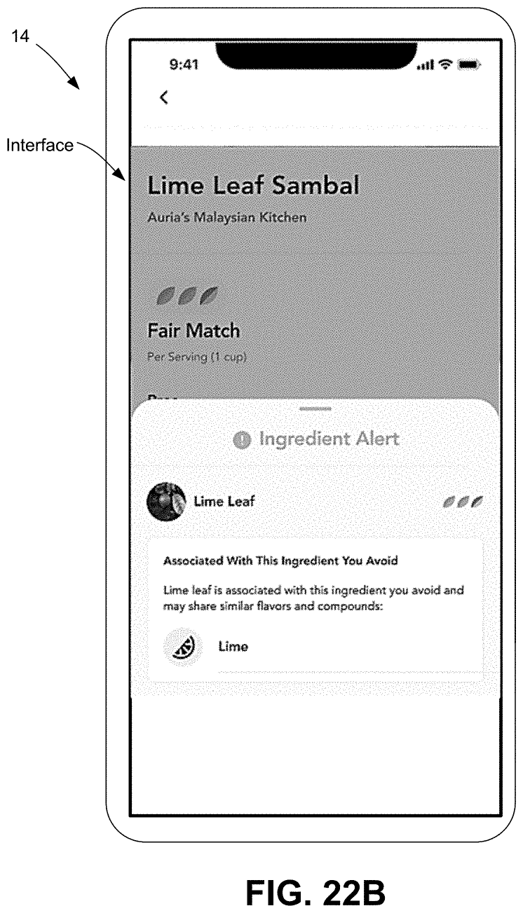

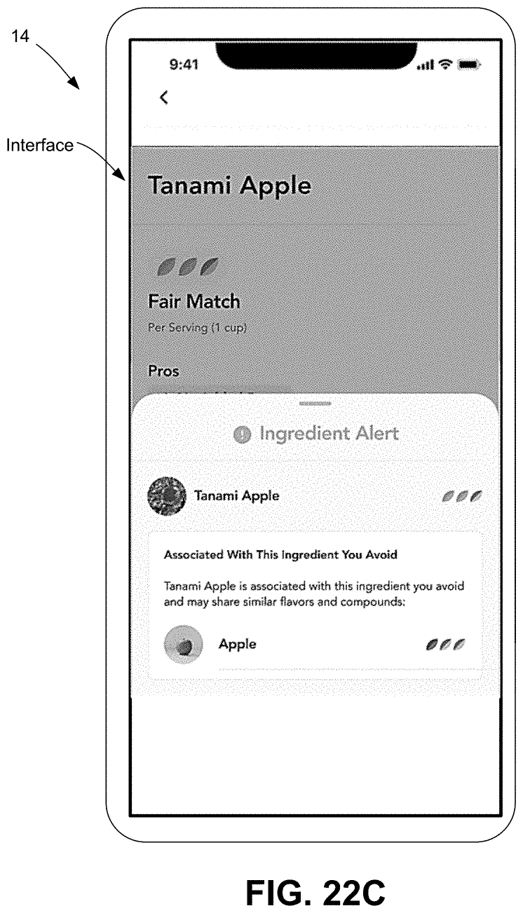

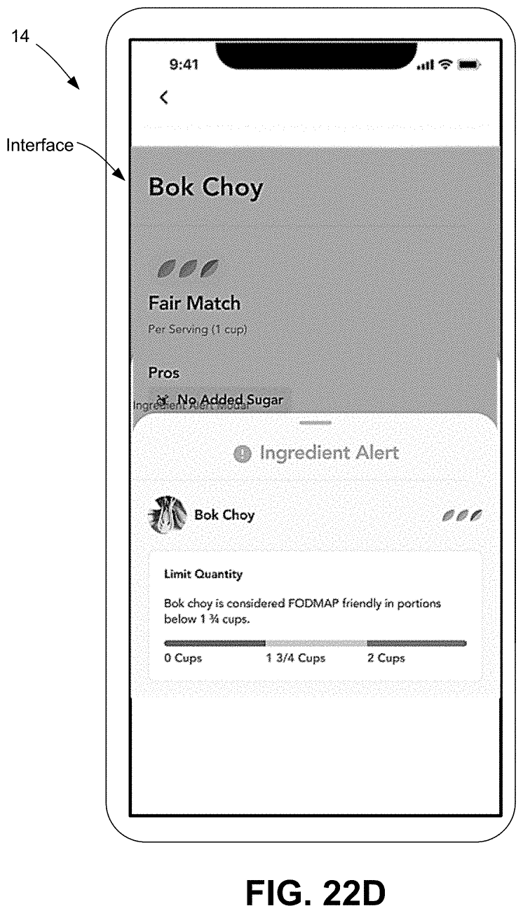

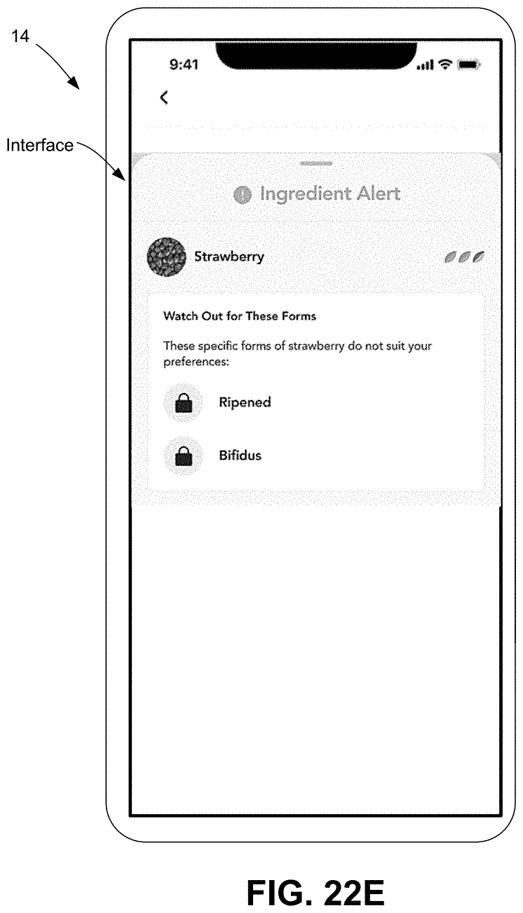

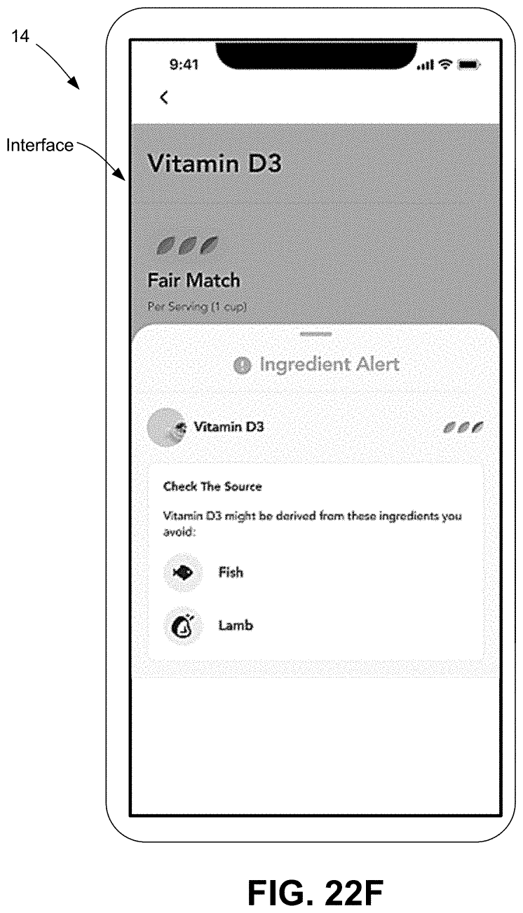

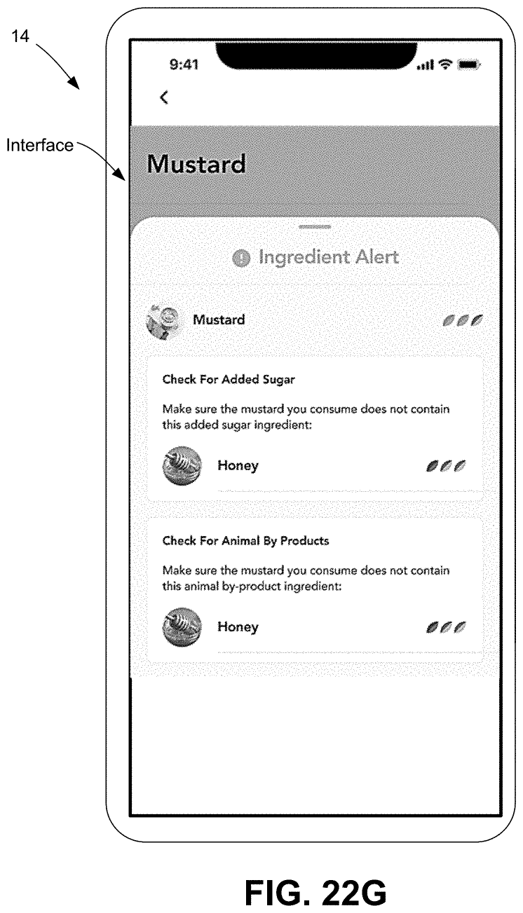

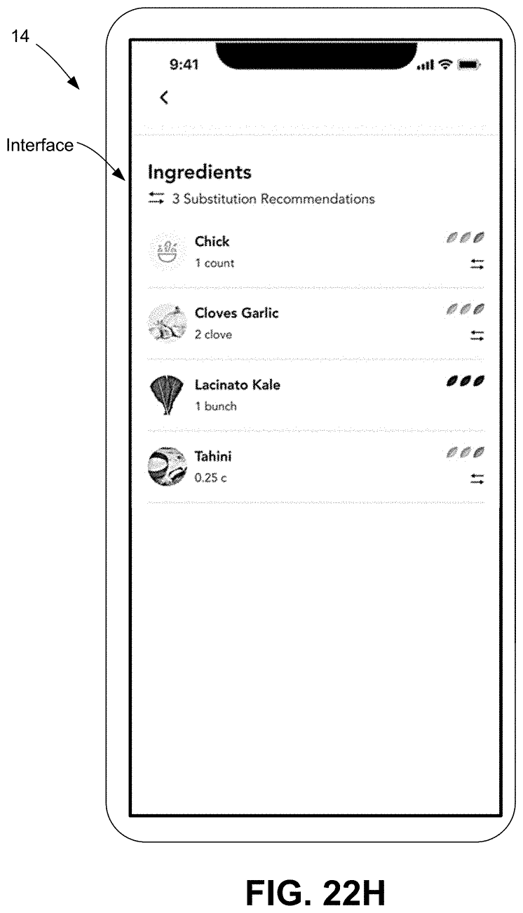

FIGS. 22A-22H are screenshots of the interface on a user's mobile device illustrating various messages associated with personalized nutritional information and recommendations via the nutrition and dietary management platform.

### Reminders:

It should be noted that, depending on users medical conditions, dietary lifestyle preferences, food intolerance, age, gender, etc, the system is configured to automatically set a baseline of dynamic recommendations for each user. Such recommendations are suggested in the mobile app during a given user's initial onboarding, as well as when a user is navigating through the available preferences. In some embodiments, if a user decides to ignore the advice, the user will be prompted with a message that essentially reminds them of the advice associated with any given food item that the user is viewing. For example, a message may state: “Did You Know? Based on your preferences, we recommend limiting the consumption of sodium. If you limit it, this item is no longer a good match for you. Do you want to update your preferences?”. Essentially, this type of reminder informs the user of the risk they are taking by ignoring the initial dietary advice provided and further provides them with the opportunity to update their preference so as to limit that risk.

### Broad Ingredients:

By nature, broad ingredients are generic, like “fruit” or “spice” or “red meat”, hence they carry a lot of tags that have an associated probability of 0.5 that could trigger a fair score if one of those tags are in the user's avoid list. As such, a user may see a message like: “There are variants of red meat that consist of or contain these ingredient categories you avoid: Beef, Lamb”. For example, FIG. 22A shows a message in which a user is advised that a certain recipe or meal may contain red meat, a broad category food items, which may include certain types of red meat that the user should avoid or has excluded based on their user attributes and preferences.

Partial Fit Sssociated with a Food Item in a User's Avoid List:

For example, a lime leaf is associated with lime, and if a user excludes lime from their diet, lime leaf will produce a fair status by association. Note that lime will produce a poor status, regardless if it comes with leaf or not. A user may see a message like: “Lime leaf is associated with this ingredient you avoid and may share similar flavors and compounds: Lime”. For example, FIG. 22B shows a message in which a user is advised that a specific food item in a given recipe or meal is associated with a food item that the user should avoid or has excluded from their diet based on their user attributes and preferences.

### Similar/Close Match Food Items:

For example, if a user excludes apples from their diet (i.e. apple is in the avoid list) and they look at a similar/close match ingredient such as a “tanami apple”. Because that ingredient shares enough similarities with apples, it will produce at best fair status. A user may see a message like: “Tanami Apple is associated with this ingredient you avoid and may share similar flavors and compounds: Apple”. For example, FIG. 22C shows a message in which a user is advised that a specific food item is similarly matched with a food item that the user should avoid or has excluded from their diet based on their user attributes and preferences.

### Food Item Quantity—Estimated Quantity:

For some food items, like products, the platform 100 can estimate the quantity of ingredients, resulting in estimating some inferred nutrients or some custom nutrients. Because those estimations carry some errors, the platform 100 can convey that information to our users when that food item has a fair status.

### Food Item Quantity—Ingredient Quantity for Specific Compounds:

For example FODMAP, used in specific diets, like the low FODMAP diet. The low FODMAP diet is not binary: it does not exclude completely some food, but instead it limits them above a certain quantity and completely excludes them above a high quantity. In the case of a fair status, a user may see a message like: “Bok choy is considered FODMAP friendly in portions below 1¾ cups.” For example, FIG. 22D shows a message in which a user is advised that a specific quantity of food item is recommended for a user preferred dietary lifestyle or therapeutic plan.

Food Item Quantity—Ingredient Quantity for Specific Compounds with a Reintroduction Phase:

Some diets are recommended for a specific amount of time. For example the low FODMAP diet is recommended for 4-6 weeks. After that grace period, users are asked to reintroduce some previously limited/avoided ingredients one by one to see which ingredients cause the medical condition symptoms. During that reintroduction phase, a user may see a message like: “Bok choy is considered sorbitol friendly in portions below 1¾ cups.” For example, FIG. 22D shows a message in which a user is advised that a specific quantity of food item is recommended for a user preferred dietary lifestyle or therapeutic plan.

### Food Item Form:

The SCD diet for example allows users to eat fresh bananas, but not ripped ones. Depending on how the ingredient is cut, processed, or aged, a user may see a message like “These specific forms of strawberry do not suit your preferences: ripened, bifidus”. For example, FIG. 22E shows a message in which a user is advised that certain forms a of specific food item is not recommended for a user preferred dietary lifestyle or therapeutic plan.

### Nutrient Source:

Some nutrients are highly correlated to their food item source, especially when they are offered in the form of concentrate or extracts, hence producing a fair status. A user may see a message like: “Make sure the Vitamin D3 used to prepare this dish is not derived from these ingredients you avoid: Fish, Lamb”. For example, FIG. 22F shows a message in which a user is cautioned that certain nutrients (i.e., vitamins, minerals, or the like) may have a high correlation to their food item source that the user should avoid or has excluded from their diet based on their user attributes and preferences.

### Food Items may Contain . . . :

Some sub-products, especially generic ones like broad sub-products, have a list of ingredients they potentially contain. For example a generic tortilla may contain wheat. An extreme example would be “pizza”, because you can put pretty much everything on a pizza. This “may contain” list produces by default tags with probability between 0-0.5 that can cause a fair status if one of the tags is in the avoid list. Or similarly if an ingredient in the avoid list is also in the may contain list. A user may see a message like: “Make sure the citric acid you consume does not contain this ingredient: Citrus”. For example, FIG. 22G shows a message in which a user is advised that a specific food item or meal may contain certain ingredients/sub-products that the user should avoid or has excluded from their diet based on their user attributes and preferences.

### Food Recommendation/Substitution

In order to empower users through their dietetic journey, the recommendation/prediction engine 114 of the platform 100 does not simply tell them they cannot have some food items. The platform 100 can also recommend alternatives based on similar food items that pass their diet (i.e. that produce a good status). Similarity is based on various food item attributes, such as nutritional info, tags, ingredient list (for sub-product/product and recipes only), food item name, etc. The AI engine 112 of the platform 100 can train a machine learning (ML) similarity engine from a training set collected in-house, and used the trained importance weights of each food attribute to store the food items in our database with associated inverted indexes, and to rank alternative food items in our substitution module. Learning the weights of each attribute is based on the paper “Local Learning Based Feature Selection for High Dimensional Data Analysis” from Yijun Sun, Sinisa Todorovic, and Steve Goodison.

In addition, the recommendation/prediction engine 114 of the platform 100 can collect for each additive, ingredient and sub-products a list of potential substitutes, already pre-ranked (the first substitute in the list being the best to be chosen, provided it complies with the diet. If not the second one is chosen, etc). That also allows the platform 100 to recommend recipes to a user with a bunch of pre-substituted ingredients that normally would not comply with the user's diet. FIG. 22H shows a message in which a user is provided with potential substitute ingredients to be used so as to avoid any potential ingredients/sub-products identified that the that the user should avoid or has excluded from their diet based on their user attributes and preferences.

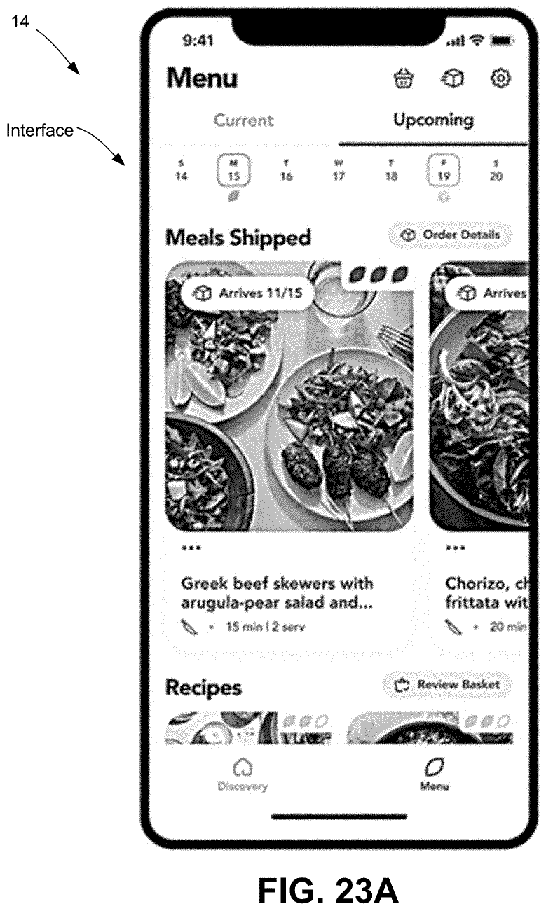

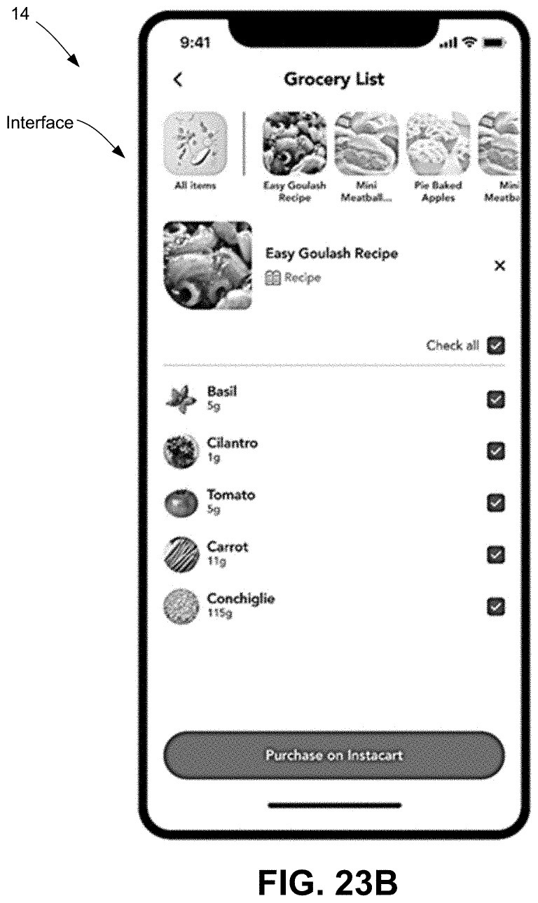

FIGS. 23A and 23B show an interface in which a user can plan their meals for any given day (see FIG. 23A) and further select specific food items to be purchased and delivered via a participating third-party service (see FIG. 23B).

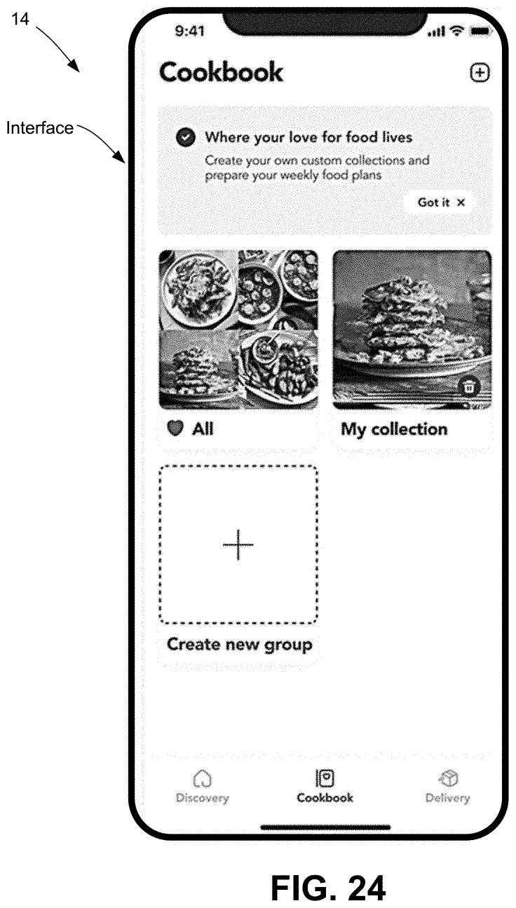

FIG. 24 shows an exemplary interface providing a user with the ability to save recipes.

Accordingly, the nutrition and dietary management platform is configured to provide a user with personalized nutritional information and recommendations based on the AI and big data analytics described above. For example, in some instances, the platform is configured to automatically provide a user with recommended food items, recipes, meal plans, and the like, based on a user set preference (i.e., the user may set a preference for the platform to automatically generate a daily, weekly, and/or monthly suggested meal plan). In some embodiments, a user may directly interact with the platform to find a given food item, recipe, or meal plan. For example, a user may use their smartphone to take a photograph of a menu item or scan a food item barcode. In turn, the system of the present invention is configured to identify the menu item (and all related food items in that menu item) or the food item and provide the user (via the platform interface) with an indication as to whether such foods adhere to the individualized nutritional preferences and, in the event that such foods do not adhere to the preferences, the system is configured to provide alternative options to choose.

Accordingly, the nutritional guidance system of the present invention, notably the nutrition and dietary management platform, better connects a user with a vast amount of nutritional data, thereby making it easier for users to make better dietary choices for every meal. The platform provides an intuitive means allowing for a user to search through a large database of ingredients to find the food items, meals, and recipes that best match a user's preferences and fit within their lifestyle. As such, the system of the present invention is able to assist a user with managing their diet and further support therapeutic nutritional plans such as keto, gluten-free, and Low-FODMAP with tools that may assist in treating or managing chronic diseases with optimal nutrition.

As used in any embodiment herein, the term “module” and/or “engine” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for providing personalized nutrition services, the system comprising:
a computing system configured to communicate with one or more user-associated computing devices over a network, the computing system comprising a hardware processor coupled to non-transitory, computer-readable memory containing instructions executable by the processor to cause the computing system to:
generate a food graph based on attributes associated with food items extracted from one or more data sources;
receive, from a computing device, user input based on user interaction with a graphical user interface (GUI) of the computing device;
generate a user graph based, at least in part, on said user input;
command a trained artificial intelligence (AI) engine to build mappings between the food graph and the user graph; and
autonomously generate one or more dietary predictions, dietary recommendations, and/or dietary modifications based, at least in part, on the mappings established between the food and user graphs.

2. The system of claim 1, wherein the computing system is further configured to autonomously tag food items with at least some of the attributes based on an initial set of attributes for the food items in the food graph.

3. The system of claim 1, wherein the computing system is further configured to detect, via the trained artificial intelligence engine, missing, hidden, and/or incorrect attribute values.

4. The system of claim 3, wherein the computing system is further configured to add or correct the missing, hidden, and/or incorrect attribute values.

5. The system of claim 1, wherein the computing system is further configured to generate food similarity metrics between the food items in the food graph based on the attributes.

6. The system of claim 1, wherein the computing system is further configured to generate user similarity metrics based on the user graph.

7. The system of claim 1, wherein the one or more dietary predictions, dietary recommendations, and dietary modifications include at least one of: a personalized diet for the user; a meal plan for the user; food recommendations for the user; restaurant recommendations for the user; recipe recommendations for the user; and product recommendations for the user.

8. The system of claim 1, wherein the user input comprises user attributes.

9. The system of claim 8, wherein the user attributes include at least one of: user biometric data, user demographic data, user food and/or symptom logs; user medical conditions; and user lab results.

10. The system of claim 9, wherein the user biometric date comprises user DNA and/or user microbiome data.

11. A method for providing personalized nutrition services, the method comprising:
generating a food graph based on attributes associated with food items extracted from one or more data sources;
receiving, from a computing device, user input based on user interaction with a graphical user interface (GUI) of the computing device;
generating a user graph based on said user input;
commanding a trained artificial intelligence (AI) engine to build mappings between the food graph and the user graph; and
autonomously generating one or more dietary predictions, dietary recommendations, and/or dietary modifications based, at least in part, on the mappings established between the food and user graphs.

12. The method of claim 11, further comprising autonomously tagging food items with at least some of the attributes based on an initial set of attributes for the food items in the food graph.

13. The method of claim 11, further comprising detecting, via the trained artificial intelligence engine, missing, hidden, and/or incorrect attribute values.

14. The method of claim 13, further comprising adding or correcting the missing, hidden, and/or incorrect attribute values.

15. The method of claim 11, further comprising generating food similarity metrics between the food items in the food graph based on the attributes.

16. The method of claim 11, further comprising generating user similarity metrics based on the user graph.

17. The method of claim 11, wherein the one or more dietary predictions, dietary recommendations, and dietary modifications include at least one of: a personalized diet for the user; a meal plan for the user; food recommendations for the user; restaurant recommendations for the user; recipe recommendations for the user; and product recommendations for the user.

18. The method of claim 11, wherein the user input comprises user attributes.

19. The method of claim 18, wherein the user attributes include at least one of: user biometric data, user demographic data, user food and/or symptom logs; user medical conditions; and user lab results.

20. The method of claim 19, wherein the user biometric date comprises user DNA and/or user microbiome data.
